(19) United States
(12) Patent Application Publication
(10) Pub. No.: US 2023/0218544 A1
(43) Pub. Date: Jul. 13, 2023

(54) TREATMENT METHODS AND FORMULATIONS

(71) Applicant: Curtin University, Bentley, Western Australia (AU)

(72) Inventors: Hani Al-Salami, Bentley, Western Australia (AU); Armin Mooranian, Bentley, Western Australia (AU); Daniel Brown, Bentley, Western Australia (AU); Jafri Kuthubutheen, Bentley, Western Australia (AU)

(21) Appl. No.: 18/001,487

(22) PCT Filed: Jun. 11, 2021
(86) PCT No.: PCT/AU2021/050605

§ 371 Date: Dec. 12, 2022

(30) Foreign Application Priority Data
Jun. 11, 2020 (AU) 2020901933

(51) Int. Cl. A61K 31/10; A61K 31/56; A61P 27/16; A61K 9/00

(52) U.S. Cl. CPC A61K 31/10; A61K 31/56; A61P 27/16; A61K 9/0046

(57) ABSTRACT

The present invention relates to methods for preventing, reducing, or treating the incidence and/or severity of a disorder caused by stress-induced cellular damage in the ear of a subject, such as vestibular disorders, hearing impairment, and conditions related to hair cell degeneration or death. Specifically, this invention pertains to formulations comprising probucol or a probucol ester, a bioavailability-enhancing compound comprising a bile acid, and optionally a carrier. The administration of these formulations can treat hearing loss and/or hair cell degeneration or death.

The present application claims priority from Australian provisional patent application no. 2020901933 filed 11 Jun. 2020. The entire contents of Australian provisional patent application no. 2020901933 are incorporated herein by reference.

### FIELD OF THE INVENTION

The present invention relates to methods for preventing, reducing or treating the incidence and/or severity of a disorder caused by stress-induced cellular damage in the inner or middle ear of a subject, such as vestibular disorders, hearing impairment, and conditions related to hair cell degeneration or hair cell death.

The present invention also relates to pharmaceutical formulations for preventing, reducing or treating the incidence and/or severity of a disorder caused by stress-induced cellular damage in the middle or inner ear of a subject, such as vestibular disorders, hearing impairment, and conditions related to hair cell degeneration or hair cell death.

### BACKGROUND

Stress-induced cellular damage in the inner and middle ear can cause a variety of different vestibular and/or hearing disorders.

The peripheral vestibular system includes the parts of the inner ear that help control balance and eye movements. Vestibular disorders or symptoms of vestibular disorders include vertigo, dizziness, imbalance, spatial disorientation, and vision disturbance.

Hearing impairment affects half of the adult population worldwide, and is growing annually at an increasing rate, due to an aging population. It is also a condition that affects children as a result of congenital disorders or acquired conditions that can result in varying degrees of hearing loss. Hearing loss is reported to have serious consequences to the general health of patients, such as mental health, and have been linked to dementia and neurodegeneration. Other than age-related hearing loss (often termed “presbycusis”), hearing loss can, inter alia, be induced by acoustic noise trauma, surgical or physical trauma, viral or bacterial infections (as well as other infective organisms), genetic disorders, and/or be drug-induced (e.g., chemotherapy-induced ototoxicity).

Hearing and balance impairment often involve the loss of sensory or neural cells, such as cochlear and vestibular hair cells and afferent neurons, but can include damaged accessory structures such as stria vascularis or cells in the spiral ligament, supporting cells (cells supporting hair cells), microvasculature endothelium, or the endolymphatic duct and sac. This cellular loss or damage can either be due to the primary insult (e.g. direct cell loss caused by noise trauma or a virus), though in many cases involves progressive cellular damage due to molecular mechanisms bringing about cellular apoptosis, driven by high levels of free-radicals, oxidative stress, and pro-inflammatory cytokines. To date, treatments aimed at suppressing the progressive loss of sensorineural or accessory cells in the inner ear have shown limited success.

Ototoxicity induced hearing loss rates between 23% and 50% in adults with cisplatin, 63% with aminoglycosides and 6-7% with furosemide. There are over 600 categories of drugs which have the potential to cause ototoxicity. Ototoxicity is considered an otologic urgency because there is less recovery of functional damage when a treatment plan is not implemented promptly.

It would be advantageous to provide new treatments that are able to normalise these molecular disturbances associated with stress-induced cellular damage in the middle or inner ear, which lead to vestibular disorders and hearing impairment. It would also be advantageous to provide effective methods for the prevention and subsequent treatment of stress-induced cellular damage in the middle or inner ear, which allow for immediate as well as long term maintenance of preventive and/or therapeutic effects.

All references, including any patents or patent applications cited in this specification, are hereby incorporated by reference.

It will be understood that, although a number of prior art publications are referred to herein, this reference does not constitute an admission that any of these publications form part of the common general knowledge in the art, in Australia or in any other country.

### SUMMARY OF THE INVENTION

In various aspects, the present invention provides the following items:

1. A method for preventing, reducing or treating the incidence and/or severity of a disorder caused by stress-induced cellular damage in the middle or inner ear of a subject, the method comprising administering to the subject a therapeutically effective amount of a compound selected from an antioxidant, an anti-inflammatory agent, and an epithelial cell protective agent.
2. A method for preventing, reducing or treating the incidence and/or severity of a vestibular disorder or hearing impairment in a subject, the method comprising administering to the subject a therapeutically effective amount of a compound selected from an antioxidant, an anti-inflammatory agent, and an epithelial cell protective agent.
3. A method for preventing, reducing or inhibiting hair cell degeneration or hair cell death in a subject, the method comprising administering to the subject a therapeutically effective amount of a compound selected from an antioxidant, an anti-inflammatory agent, and an epithelial cell protective agent.
4. Use of a compound selected from an antioxidant, an anti-inflammatory agent, and an epithelial cell protective agent in the manufacture of a medicament for preventing, reducing or treating the incidence and/or severity of a disorder caused by stress-induced cellular damage in the middle or inner ear of a subject.
5. Use of a compound selected from an antioxidant, an anti-inflammatory agent, and an epithelial cell protective agent in the manufacture of a medicament for preventing, reducing or treating the incidence and/or severity of a vestibular disorder or hearing impairment in a subject.
6. Use of a compound selected from an antioxidant, an anti-inflammatory agent, and an epithelial cell protective agent in the manufacture of a medicament for preventing, reducing or inhibiting hair cell degeneration or hair cell death in a subject.
7. A compound selected from an antioxidant, an anti-inflammatory agent, and an epithelial cell protective agent for use in preventing, reducing or treating the incidence and/or severity of a disorder caused by stress-induced cellular damage in the middle or inner ear of a subject.
8. A compound selected from an antioxidant, an anti-inflammatory agent, and an epithelial cell protective agent for use in preventing, reducing or treating the incidence and/or severity of a vestibular disorder or hearing impairment in a subject.
9. A compound selected from an antioxidant, an anti-inflammatory agent, and an epithelial cell protective agent for use in preventing, reducing or inhibiting hair cell degeneration or hair cell death in a subject.
10. A pharmaceutical formulation comprising:
(i) an active ingredient;
(ii) an agent that enhances the bioavailability of the active ingredient, wherein the agent comprises an amphiphilic compound of the formula (I):

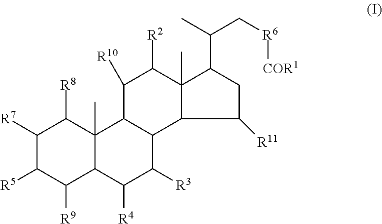

wherein:
   each R¹, R², R³, R⁴, R⁵, R⁷, R⁸, R⁹, R¹⁰ and R¹¹ is independently selected from H, substituted or unsubstituted C₁₋₃₀ alkyl, substituted or unsubstituted C₂₋₃₀ alkenyl, substituted or unsubstituted C₂₋₃ alkynyl, substituted or unsubstituted C₃₋₃₀ cycloalkyl, substituted or unsubstituted aryl, substituted or unsubstituted arylalkyl, substituted or unsubstituted heteroaryl, or GL; wherein when R¹, R², R³, R⁴, R⁵, R⁷, R⁸, R⁹, R¹⁰ or R¹¹ is substituted, the substituent is independently selected from OH, F, SH, ═O, ═S, Cl, Br, SC₁₋₆ alkyl, C₁₋₆ alkyl or C₁₋₆ alkoxy;
   G is absent or is selected from O, S, PL, CL₂, or NL;
   each L is independently selected from H, a metallic ion, substituted or unsubstituted C₁₋₃₀ alkyl, substituted or unsubstituted C₂₋₃₀ alkenyl, substituted or unsubstituted C₂₋₃₀ alkynyl, substituted or unsubstituted C₃₋₃₀ cycloalkyl, a substituted or unsubstituted benzyl radical, —CH₂CO₂H, or —(CH₂)₂SO₃H; wherein when L is substituted, the substituent is independently selected from F, SH, OH, ═O, ═S, Cl, Br, SC₁₋₆ alkyl, C₁₋₆ alkyl or C₁₋₆ alkoxy; or when L is bonded to R¹, L may be an amino acid; and
   R⁶ is —(CH₂)ₙ— wherein n is 0 to 12;
   or a stereoisomer thereof, or a pharmaceutically acceptable salt thereof; and
(iii) optionally a pharmaceutically acceptable carrier;
wherein the formulation is formulated to be administered to the middle or inner ear.
   11. A method for preventing, reducing or treating the incidence and/or severity of a disorder caused by stress-induced cellular damage in the middle or inner ear of a subject, the method comprising administering to the subject an effective amount of the formulation according to item 10.
   12. A method for preventing, reducing or treating the incidence and/or severity of a vestibular disorder or hearing impairment in a subject, the method comprising administering to the subject an effective amount of the formulation according to item 10.
   13. A method for preventing, reducing or inhibiting hair cell degeneration or hair cell death in a subject, the method comprising administering to the subject an effective amount of the formulation according to item 10.
   14. Use of an active agent in the preparation of a formulation according to item 10 for preventing, reducing or treating the incidence and/or severity of a disorder caused by stress-induced cellular damage in the middle or inner ear of a subject.
   15. Use of an active agent in the preparation of a formulation according to item 10 for preventing, reducing or treating the incidence and/or severity of a vestibular disorder or hearing impairment in a subject.
   16. Use of an active agent in the preparation of a formulation according to item 10 for preventing, reducing or inhibiting hair cell degeneration or hair cell death in a subject.
   17. A formulation according to item 10 for use in preventing, reducing or treating the incidence and/or severity of a disorder caused by stress-induced cellular damage in the middle or inner ear of a subject.
   18. A formulation according to item 10 for use in preventing, reducing or treating the incidence and/or severity of a vestibular disorder or hearing impairment in a subject.
   19. A formulation according to item 10 for use in preventing, reducing or inhibiting hair cell degeneration or hair cell death in a subject.

### BRIEF DESCRIPTION OF THE FIGURES

Embodiments of the present invention will be further described, by way of example only, with reference to the accompanying figures, in which:

FIG. 1: shows the amount of probucol that had diffused or permeated into the cochlea (“Round Window turn” indicates probucol levels straight after (immediately past) the round window of the cochlea where it was expected that most of the drug would be retained, while “Deep Layers” indicates probucol levels deep in the cochlea) in C57 healthy mice (left side), and SAMP8 aged mice (right side).

FIG. 2: shows detection thresholds (and standard deviations) for Compound Action Potentials (CAPs) evoked by 18, 14, 10, 6 and 2 kHz tone-bursts, recorded with a AgCl electrode on the Round Window, before the application of probucol, and 1.5 hour after probucol nanoparticle formulation had been applied to the round window membrane. The CAP thresholds are given in units of dB SPL (Sound Pressure Level). There was no statistically significant difference in CAP thresholds before or 1.5 hours after probucol nanoparticle formulation.

FIG. 3: shows: A) Average round window CAP thresholds, either before (baseline) otoxicity, or after ototoxicity treatment but with (probucol) and without (control) probucol therapy (with Standard Error Bars); and B) Decibel difference between round window CAP thresholds of control and probucol treated cochleae, after ototoxicity. n=8 animals.

FIG. 4: shows the cytotoxicity in inner ear sensory cells (HEI-OC1) exposed to cisplatin. (A): images of cells treated with increased of cisplatin. (B): cell counts (%) after 24 hours incubation. (C): images of Myo7a and Nestin expression at 33° C. and 10% CO₂ atmosphere condition. (D): cell growth curve of untreated cells, over 14 days (at two different temperatures). Data are mean±SD.

FIG. 5: shows the cell viability with cisplatin and probucol. (A): images of control (cells treated with cisplatin) and probucol treated cells. (B): Relative (ratio) increase in cell count due to probucol incorporation. Data are mean±SD.

FIG. 6: shows round window Compound Action Potential thresholds of guinea pig model of ototoxicity post probucol treatment. (A): round window Compound Action Potential thresholds, before ototoxicity (normal), after ototoxicity (ototoxicity) or after ototoxicity treated with probucol (ototoxicity+PB) groups. (B): Decibel difference between the thresholds of ototoxicity and ototoxicity+PB groups. N=8 animals and data are mean±SD.

FIG. 7: shows the identification of spiral ganglion neurons (SGNs) cell bodies from an intact whole cochlea of a guinea pig, scanned using light sheet microscopy (LSM). Rosenthal's canal was manually segmented using Arivis 4D software, and an inbuilt blob-detection algorithm was used to detect SGN cells throughout the cochlea. (A): whole SGNs (yellow) of guinea pig cochlea was derived using a 3D reconstruction. (B): 2D sectional image of the cochlea showing well-defined SGN cell bodies (yellow) within Rosenthal's canal throughout the cochlea. (C): a comparison between the density of SGNs in ototoxicity, and ototoxicity+probucol group, taken from the basal, middle and apical turns of the cochlea.

FIG. 8: shows the quantification of SGN cells throughout the cochlea in the study groups. (A): 3D plot of individual locations of every detected SGN cells throughout the cochlear spiral. (B): 2D plot of SGN locations, plotted according to the Z-axis showing SGN density in the basal, middle, and apical turns. (C): a comparison of the populations of SGNs between the ototoxicity and ototoxicity+probucol groups. In the basal turn, there was a significantly larger density of SGNs in the probucol group, compared to that of ototoxicity alone group.

### DETAILED DESCRIPTION OF EMBODIMENTS OF THE INVENTION

Preferred embodiments of the present invention are described below by way of example only.

### 1. Definitions

Unless otherwise herein defined, the following terms will be understood to have the general meanings which follow. The terms referred to below have the general meanings which follow when the term is used alone and when the term is used in combination with other terms, unless otherwise indicated. Hence, for example, the definition of “alkyl” applies to “alkyl” as well as the “alkyl” portions of “haloalkyl”, “heteroalkyl”, etc.

The term “amphiphilic compound” as used herein is any compound having a hydrophilic/lipophilic balance (HLB) value between about 2 and about 20, inclusive. Preferred amphiphilic compounds have an HLB value between about 6 and about 16.

The term “alkyl” refers to a straight chain or branched chain saturated hydrocarbyl group. Unless indicated otherwise, preferred are C₁₋₆ alkyl and C₁₋₄ alkyl groups. The term “C_{x-y} alkyl”, where x and y are integers, refers to an alkyl group having x to y carbon atoms. For example, the term “C₁₋₆ alkyl” refers to an alkyl group having 1 to 6 carbon atoms. Examples of C₁₋₆ alkyl include methyl (Me), ethyl (Et), propyl (Pr), isopropyl (i-Pr), butyl (Bu), isobutyl (i-Bu), sec-butyl (s-Bu), tert-butyl (t-Bu), pentyl, neopentyl, hexyl and the like. Unless the context requires otherwise, the term “alkyl” also encompasses alkyl groups containing one less hydrogen atom such that the group is attached via two positions, i.e. divalent.

The term “alkenyl” refers to a straight chain or branched chain hydrocarbyl group having at least one double bond of either E- or Z-stereochemistry where applicable. Unless indicated otherwise, preferred are C₂₋₆ alkenyl and C₂₋₃alkenyl groups. The term “C_{x-y} alkenyl”, where x and y are integers, refers to an alkenyl group having x to y carbon atoms. For example, the term “C₂₋₆alkenyl” refers to an alkenyl group having 2 to 6 carbon atoms. Examples of C₂₋₆ alkenyl include vinyl, 1-propenyl, 1- and 2-butenyl and 2-methyl-2-propenyl. Unless the context requires otherwise, the term “alkenyl” also encompasses alkenyl groups containing one less hydrogen atom such that the group is attached via two positions, i.e. divalent.

The term “alkynyl” refers to a straight chain or branched chain hydrocarbyl group having at least one triple bond. Unless indicated otherwise, preferred are C₂₋₆ alkynyl and C₂₋₃alkynyl groups. The term “C_{x-y} alkynyl”, where x and y are integers, refers to an alkynyl group having x to y carbon atoms. For example, the term “C₂₋₆ alkynyl” refers to an alkynyl group having 2 to 6 carbon atoms. Examples of C₂₋₆ alkynyl include ethynyl, 1-propynyl, 1- and 2-butynyl, 2-pentynyl, 3-pentynyl, 4-pentynyl, 2-hexynyl, 3-hexynyl, 4-hexynyl and 5-hexynyl and the like. Unless the context indicates otherwise, the term “alkynyl” also encompasses alkynyl groups containing one less hydrogen atom such that the group is attached via two positions, i.e. divalent.

The term “C₃₋₃₀ cycloalkyl” refers to a non-aromatic cyclic hydrocarbyl group having from 3 to 30 carbon atoms. Such groups include cyclopropyl, cyclobutyl, cyclopentyl, cyclohexyl, cycloheptyl and cyclooctyl. The term “C₃₋₃₀ cycloalkyl” encompasses groups where the cyclic hydrocarbyl group is saturated such as cyclohexyl or unsaturated such as cyclohexenyl. In some embodiments, the C₃₋₃₀ cycloalkyl is C₃₋₈ cycloalkyl. C₃₋₆ cycloalkyl such as cyclopropyl, cyclobutyl, cyclopentyl and cyclohexyl are preferred.

The term “alkoxy” refers to an alkyl group as defined above covalently bound via an O linkage, such as methoxy, ethoxy, propoxy, isopropoxy, butoxy, tert-butoxy and pentoxy. Unless indicated otherwise, preferred are C₁₋₆ alkoxy, C₁₋₄ alkoxy and C₁₋₃ alkoxy groups.

The term “aryl” refers to a carbocyclic (non-heterocyclic) aromatic ring or mono-, bi- or tri-cyclic ring system. The aromatic ring or ring system is generally composed of 6 to 10 carbon atoms. Examples of aryl groups include but are not limited to phenyl, biphenyl, naphthyl and tetrahydronaphthyl. 6-Membered aryls such as phenyl are preferred.

The term “arylalkyl” or “aralkyl” refers to an arylC₁₋₆alkyl—such as benzyl.

The term “heteroaryl” is used herein to denote a heterocyclic group having aromatic character and embraces aromatic monocyclic ring systems and polycyclic (e.g. bicyclic) ring systems containing one or more aromatic rings. The term aromatic heterocyclyl also encompasses pseudoaromatic heterocyclyls. The term “pseudoaromatic” refers to a ring system which is not strictly aromatic, but which is stabilized by means of delocalization of electrons and behaves in a similar manner to aromatic rings. The term aromatic heterocyclyl therefore covers polycyclic ring systems in which all of the fused rings are aromatic as well as ring systems where one or more rings are non-aromatic, provided that at least one ring is aromatic. In polycyclic systems containing both aromatic and non-aromatic rings fused together, the group may be attached to another moiety by the aromatic ring or by a non-aromatic ring.

Examples of heteroaryl groups are monocyclic and bicyclic groups containing from five to ten ring members. The heteroaryl group can be, for example, a five membered or six membered monocyclic ring or a bicyclic structure formed from fused five and six membered rings or two fused six membered rings or two fused five membered rings. Each ring may contain up to four heteroatoms selected from nitrogen, sulphur and oxygen. The heteroaryl group can contain up to 4 heteroatoms, more typically up to 3 heteroatoms, more usually up to 2 heteroatoms. In one embodiment, the heteroaryl group contains at least one ring nitrogen atom. The nitrogen atoms in the heteroaryl group can be basic, as in the case of an imidazole or pyridine, or essentially non-basic as in the case of an indole or pyrrole nitrogen. In general, the number of basic nitrogen atoms present in the heteroaryl group, including any amino group substituents of the ring, will be less than five.

Examples of heteroaryl groups containing an aromatic ring and a non-aromatic ring include tetrahydroisoquinoline, tetrahydroquinoline, dihydrobenzothiophene, dihydrobenzofuran, 2,3-dihydro-benzo[1,4]dioxine, benzo[1,3]dioxole, 4,5,6,7-tetrahydrobenzofuran, indoline and isoindoline groups.

The term “chiral” refers to molecules which have the property of non-superimposability of the mirror image partner, while the term “achiral” refers to molecules which are superimposable on their mirror image partner.

The term “diastereomers” refers to stereoisomers with two or more centers of dissymmetry and whose molecules are not mirror images of one another.

The term “enantiomers” refers to two stereoisomers of a compound which are non-superimposable mirror images of one another. An equimolar mixture of two enantiomers is called a “racemic mixture” or a “racemate.”

The term “isomers” or “stereoisomers” refers to compounds which have identical chemical constitution, but differ with regard to the arrangement of the atoms or groups in space.

Throughout this specification, including the claims, the word “include”, or variations thereof such as “includes” or “including”, will be understood to imply the inclusion of a stated element, integer or step, or group of elements, integers or steps, but not the exclusion of any other element, integer or step, or group of elements, integers or steps.

Throughout this specification, including the claims, except where the context requires otherwise due to express language or necessary implication, the word “comprise” or variations such as “comprises” or “comprising” is used in an inclusive sense, i.e. to specify the presence of a stated element, integer or step, or group of elements, integers or steps, but not to preclude the presence or addition of a further element, integer or step, or a further group of elements, integers or steps, in various embodiments of the invention.

The term “pharmaceutically acceptable carrier” as used herein refers to a carrier or excipient or diluent that is suitable for use with humans and/or animals without undue adverse side effects (such as toxicity, irritation, and allergic response) commensurate with a reasonable benefit/risk ratio. It may be a pharmaceutically acceptable solvent, suspending agent or vehicle, for delivering active ingredients to the subject.

The term “controlled release” as used herein refers to the control of the rate and/or quantity of active ingredients delivered according to the pharmaceutical formulations of the invention. The controlled release kinetics may be prolonged or sustained release, fast or immediate release, delayed release or pulsatile drug delivery system.

The terms “individual”, “subject” and “patient” are used herein interchangeably. In certain embodiments, the subject is a mammal. Mammals include, but are not limited to, primates (including human and non-human primates). In a preferred embodiment, the subject is a human.

The term “disorder caused by stress-induced cellular damage in the middle or inner ear” as used herein is defined as any disorder which is caused by stress-induced cellular damage in the middle or inner ear. The cellular damage in the middle or inner ear may be induced by any form of insult that can increase cellular stress in the middle or inner ear via molecular disturbances, including for example, by a physical or acoustic trauma, a bacterial, viral or other type of infection, or chemical ototoxic insult, resulting in high levels of free-radicals, oxidative stress, pro-inflammatory cytokines, affecting normal cellular function in the middle inner ear, causing inflamed or damaged specialized middle or inner ear epithelia such as stria vascularis, spiral ligament, organ of Corti, endolymphatic sac, crista amplularis, otoliths, spiral ganglion, auditory nerves, along with neuronal signalling loss and general apoptosis throughout the middle or inner ear. Changes in the middle ear can affect the inner ear. For example, otitis media, a primarily middle ear problem, can result in sensorineural hearing loss and imbalance, which are both inner ear problems.

The term “hearing disorder” as used herein is defined as any disorder caused by molecular disturbances affecting the inner or middle ear and their relevant neural connections to the brain.

The term “hearing impairment” as used herein is defined as any impairment of hearing caused by molecular disturbances affecting the inner or middle ear and their relevant neural connections to the brain.

The term “hearing loss” as used herein is defined as a diminished ability to perceive sounds relative to normative levels. This may be caused either by a conductive hearing loss, sensorineural hearing loss, or a combination of both. Hearing loss includes presbycusis (age-related hearing loss).

The term “conductive hearing loss” as used herein is one where sound pressure transmission from the external to inner ear is attenuated, for example, due to an excessive build-up of earwax, glue ear, an ear infection with inflammation and fluid build-up, a perforated or defective eardrum, a skin growth in the middle ear (cholesteatoma), or a malfunction of the ossicles (bones in the middle ear).

The term “sensorineural hearing loss” as used herein is caused by dysfunction of the sensory and/or neural cells of the cochlea, and/or dysfunction of the specialized epithelia in the inner ear such as stria vacularis and cochlear supporting cells, as well as their relevant neural connections from the ear to the brain.

The term “vestibular dysfunction” as used herein is any impairment of balance or vision stabilization caused by molecular disturbances affecting the inner ear.

The term “hair cell degeneration” or “hair cell loss” as used herein refers to a gradual loss of hair cell function and integrity and/or leading ultimately to hair cell death.

The term “hair cell death” as used herein refers to apoptosis of the hair cells in the middle or inner ear.

The terms “identification of hair cell damage” and “detection of hair cell damage” are used interchangeably herein and refer to a method by which the degree of hair cell damage in the middle or inner ear may be determined. Such methods are known in the art and comprise, for example, fluorescent imaging of the hair cells. An audiogram that demonstrates loss of hearing sensitivity at moderate to high frequencies is also indicative of hair cell damage. A decrease of hearing potential with no subsequent recovery is also diagnostic of hair cell damage.

The terms “chemically induced hearing loss” and “hearing loss induced by a chemical” as used herein refer to hearing loss which is induced and/or caused by chemical agents, such as solvents, gases, paints, heavy metals, and/or medicaments which are ototoxic.

An “effective amount” or “therapeutically effective amount” is an amount sufficient to effect a beneficial or desired therapeutic effect. This amount may be the same or different from a “prophylactically effective amount”, which is an amount necessary to prevent onset of the disorder or disorder symptoms. An effective amount may be administered in one or more administrations, applications or dosages. A therapeutically effective amount of a therapeutic compound (i.e., an effective dosage) depends on the therapeutic compounds selected.

The term “treating” as used herein refers to affecting a subject, tissue or cell to obtain a desired pharmacological and/or physiological effect and includes inhibiting the condition, i.e. arresting its development; or relieving or ameliorating the effects of the condition i.e., cause reversal or regression of the effects of the condition.

The term “preventing” as used herein refers to preventing a condition from occurring in a cell, tissue or subject that may be at risk of having the condition, but does not necessarily mean that condition will not eventually develop, or that a subject will not eventually develop a condition. Preventing includes delaying the onset of a condition in a cell, tissue or subject.

The singular terms “a”, “an”, and “the” include plural referents unless the context clearly indicates otherwise. Similarly, the word “or” is intended to include “and” unless the context clearly indicates otherwise. Although methods and materials similar or equivalent to those described herein may be used in the practice or testing of this disclosure, suitable methods and materials are described below.

The abbreviation, “e.g.” is derived from the Latin exempli gratia, and is used herein to indicate a non-limiting example. Thus, the abbreviation “e.g.” is synonymous with the term “for example”.

The use of any and all examples, or exemplary language (for example, “such as”) provided with respect to certain embodiments herein is intended merely to better illuminate the application and does not pose a limitation on the scope of the application otherwise claimed. No language in the specification should be construed as indicating any non-claimed element essential to the practice of the application.

The term “about” as used herein refers to +/−up to 20% of a given measurement. For example, the term “about” refers to a value that is within plus or minus 10% or within plus or minus 5% of the recited value.

The recitation of ranges of values herein is merely intended to serve as a shorthand method of referring individually to each separate value falling within the range. Unless otherwise indicated herein, each individual value is incorporated into the specification as if it were individually recited herein.

All methods described herein may be performed in any suitable order unless otherwise indicated herein or otherwise clearly contradicted by context.

### 2. Aspects of the Present Invention

In one aspect, the present invention provides methods for preventing, reducing or treating the incidence and/or severity of a disorder caused by stress-induced cellular damage in the middle or inner ear of a subject using a compound selected from an antioxidant, an anti-inflammatory agent, and an epithelial cell protective agent, e.g. probucol. The disorder may, for example, be a vestibular disorder, hearing impairment, or a disorder related to, or caused by, hair cell degeneration or hair cell death.

In another aspect, the present invention provides pharmaceutical formulations for preventing, reducing or treating the incidence and/or severity of a disorder caused by stress-induced cellular damage in the middle or inner ear of a subject. The disorder may, for example, be a vestibular disorder, hearing impairment, or a disorder related to, or caused by, hair cell degeneration or hair cell death. The pharmaceutical formulations may include any active ingredient useful for preventing, reducing or treating the incidence and/or severity of a disorder caused by stress-induced cellular damage in the middle or inner ear. The pharmaceutical formulations may also be used as medicaments for recovery and/or therapy and/or pre-treatment for ear-associated surgery for outer/middle/inner ear implanted devices (e.g., cochlear implantation and grommets).

Active ingredients that may be usefully administered with the pharmaceutical formulation include, for example, corticosteroids, antibiotics, neurotrophins, growth factors, anti-fibrotic agents, and stem cell promoting agents. In some embodiments, the active ingredient is probucol.

Thus, in various aspects, the present invention provides methods, uses and formulations to prevent, reduce or treat the incidence and/or severity of a disorder caused by stress-induced cellular damage in the middle and inner ear, such as vestibular disorders and hearing impairment involving inner ear tissue, particularly inner ear hair cells (e.g. hair-cell loss at the apical end of the cochlea), the stria vascularis (e.g. inflamed and damaged stria vascularis), and associated auditory nerves including neuronal signalling loss and apoptosis throughout the middle or inner ear. Hearing impairment includes those conditions that lead to permanent hearing loss where a reduced number of hair cells may be responsible and/or decreased hair cell function. Hearing impairment includes hearing impairment arising as an unwanted side-effect of ototoxic therapeutic drugs, such as cisplatin and its analogues, aminoglycoside antibiotics, salicylate and its analogues, or loop diuretics.

In some embodiments, the present invention relates to maintaining, inducing, promoting, or enhancing the viability or regeneration of middle or inner ear cells, particularly middle or inner ear supporting cells and hair cells. The inventors believe that probucol has the potential to aid recovery of hearing loss and relevant cell functions.

Whereas mechanical devices simply increase the volume, the methods and formulations of the invention can advantageously restore the frequency response of the ear and, unlike surgical interventions, the methods and formulations of the invention are non-invasive.

2.1. Methods of the Invention Using an Antioxidant, an Anti-Inflammatory Agent, or an Epithelial Cell Protective Agent (e.g. Probucol)

In one aspect, the present invention provides a method for preventing, reducing or treating the incidence and/or severity of a disorder caused by stress-induced cellular damage in the middle or inner ear of a subject, the method comprising administering to the subject a therapeutically effective amount of a compound selected from an antioxidant, an anti-inflammatory agent, and an epithelial cell protective agent.

In a further aspect, the present invention provides the use of a compound selected from an antioxidant, an anti-inflammatory agent, and an epithelial cell protective agent in the manufacture of a medicament for preventing, reducing or treating the incidence and/or severity of a disorder caused by stress-induced cellular damage in the middle or inner ear of a subject.

In some embodiments, the disorder caused by stress-induced cellular damage is a vestibular disorder. In some embodiments, the disorder caused by stress-induced cellular damage is hearing impairment, such as hearing loss.

Thus, in another aspect, the present invention provides a method for preventing, reducing or treating the incidence and/or severity of a vestibular disorder or hearing impairment in a subject, the method comprising administering to the subject a therapeutically effective amount of a compound selected from an antioxidant, an anti-inflammatory agent, and an epithelial cell protective agent.

In a further aspect, the present invention provides the use of a compound selected from an antioxidant, an anti-inflammatory agent, and an epithelial cell protective agent in the manufacture of a medicament for preventing, reducing or treating the incidence and/or severity of a vestibular disorder or hearing impairment in a subject.

In some embodiments, the stress-induced cellular damage leads to hair cell degeneration and/or hair cell death. Thus, in some embodiments, the disorder caused by stress-induced cellular damage is a disorder caused by hair cell degeneration and/or hair cell death.

Thus, in another aspect, the present invention provides a method for preventing, reducing or inhibiting hair cell degeneration or hair cell death in a subject, the method comprising administering to the subject a therapeutically effective amount of a compound selected from an antioxidant, an anti-inflammatory agent, and an epithelial cell protective agent.

In a further aspect, the present invention provides the use of a compound selected from an antioxidant, an anti-inflammatory agent, and an epithelial cell protective agent in the manufacture of a medicament for preventing, reducing or inhibiting hair cell degeneration or hair cell death in a subject.

In some embodiments, the compound selected from an antioxidant, an anti-inflammatory agent, and an epithelial cell protective agent is a poorly water-soluble and/or highly lipophilic drug.

In some embodiments of the above aspects, the compound is selected from probucol, succinobucol (4-[2,6-ditert-butyl-4-[2-(3,5-ditert-butyl-4-hydroxyphenyl)sulfanylpropan-2-ylsulfanyl]phenoxy]-4-oxobutanoic acid), probucol analogues, statins, and gliclazide.

Statins are a class of cholesterol-reducing drugs which are available commercially. Statins suitable for use in the invention include, for example, atorvastatin and simvastatin.

In some embodiments, the compound is probucol.

Probucol (2,6-ditert-butyl-4-[2-(3,5-ditert-butyl-4-hydroxyphenyl)sulfanylpropan-2-ylsulfanyl]phenol) is a hydrophobic compound that has been widely prescribed as a lipid-lowering or anti-hypercholesteremia drug. It has no reported side effects related to causing hearing loss (i.e. probucol, as used, is not an ototoxic drug). Probucol is available commercially. Probucol has antioxidative properties, and this may be due to its phenolic structure. Probucol is a potent oxygen radical scavenger that can serve as a powerful anti-inflammatory agent to suppress oxidant induced tissue injury, in addition to being a cholesterol reducing and anti-atherogenic drug. Because glutathione peroxidase (GPx) plays a crucial role in preventing oxidative stress, the pharmacological use of its mimetics has been proposed as a strategy to treat oxidative stress-related pathological condition. In some studies, the protective effects of probucol were paralleled by significant increases in GPx activity.

The inventors have now found new evidence that suggests that probucol and similar antioxidant, anti-inflammatory and endothelial cell protective compounds have potential desirable effects contributing to preventing, reducing or treating the incidence and/or severity of a disorder caused by stress-induced cellular damage in the middle or inner ear. The cellular damage in the middle or inner ear may, for example, be induced or caused by stress due to high levels of free radicals, oxidative stress, pro-inflammatory cytokines, noise-induced stress, chemically-induced stress, infection by viruses or bacteria or other infective agents, physical trauma, molecular disturbances affecting the cochlea, inflamed or damaged stria vascularis, neuronal signalling loss and apoptosis throughout the middle ear or inner ear. The cellular damage in the middle or inner ear may, for example, lead to hair cell degeneration and/or hair cell death.

Without wishing to be bound by theory, the inventors believe that probucol may act as an antioxidant, an anti-inflammatory agent, and/or an epithelial cell protective agent when delivered to the middle or inner ear, and may thereby exert protective effects on hair cells, the stria vascularis and VIIIth nerve neurons. The inventors believe that probucol, interalia, may aid to restore the viability of hair cells, via reduced cellular damage throughout the middle or inner ear.

Changes in the middle ear can affect the inner ear. There are also conditions which are confined primarily to the middle ear, but which can have inner ear effects. For example, otitis media, which is an infective/inflammatory condition primarily of the middle ear, can result in sensorineural hearing loss (or sensorineural deafness) and imbalance, which are both inner ear problems even after the middle ear infection has settled (due to spread of infection from the middle ear to the inner ear via the round window, a structure which is at the boundary between the middle ear and inner ear). Another example is some antibiotic ear drops are ototoxic when given in the middle ear, because the drops also enter the inner ear. A further example is a resident middle ear infection (e.g. biofilm) that could cause inner ear damage due to free-radical migration.

The inventors believe that probucol and similar antioxidant, anti-inflammatory and endothelial cell protective compounds, delivered/administered to the middle ear, whilst having effects on the inner ear, may also modulate disease processes within the middle ear and affect outcomes in the inner ear. Probucol and similar antioxidant, anti-inflammatory and endothelial cell protective compounds may be able to ameliorate conditions in the middle ear if given in the middle ear by its effect not only on the inner ear but potentially on structures and tissues in the middle ear such as the round window membrane. In particular embodiments, the compound is probucol.

Thus, in one aspect, the present invention provides a method for preventing, reducing or treating the incidence and/or severity of a disorder caused by stress-induced cellular damage in the middle or inner ear of a subject, the method comprising administering to the subject a therapeutically effective amount of probucol.

In a further aspect, the present invention provides the use of probucol in the manufacture of a medicament for preventing, reducing or treating the incidence and/or severity of a disorder caused by stress-induced cellular damage in the middle or inner ear of a subject.

In some embodiments, the disorder caused by stress-induced cellular damage is a vestibular disorder. In some embodiments, the disorder caused by stress-induced cellular damage is hearing impairment, such as hearing loss.

Thus, in another aspect, the present invention provides a method for preventing, reducing or treating the incidence and/or severity of a vestibular disorder or hearing impairment in a subject, the method comprising administering to the subject a therapeutically effective amount of probucol.

In a further aspect, the present invention provides the use of probucol in the manufacture of a medicament for preventing, reducing or treating the incidence and/or severity of a vestibular disorder or hearing impairment in a subject.

In some embodiments, the stress-induced cellular damage leads to hair cell degeneration and/or hair cell death. Thus, in some embodiments, the disorder caused by stress-induced cellular damage is a disorder caused by hair cell degeneration and/or hair cell death.

Thus, in another aspect, the present invention provides a method for preventing, reducing or inhibiting hair cell degeneration or hair cell death in a subject, the method comprising administering to the subject a therapeutically effective amount of probucol.

In a further aspect, the present invention provides the use of probucol in the manufacture of a medicament for preventing, reducing or inhibiting hair cell degeneration or hair cell death in a subject.

In a further aspect, the present invention provides pharmaceutical formulations comprising a compound selected from an antioxidant, an anti-inflammatory agent, and an epithelial cell protective agent (e.g. probucol) which are suitable for use in the methods and uses of the invention. Pharmaceutical formulations comprising a compound selected from an antioxidant, an anti-inflammatory agent, and an epithelial cell protective agent (e.g. probucol) suitable for use in the methods and uses of the invention are described in further detail below and in Section 2.2 below.

Thus, in various aspects, the present invention provides methods, uses and formulations employing a compound selected from an antioxidant, an anti-inflammatory agent, and an epithelial cell protective agent (e.g. probucol) to prevent, reduce or treat the incidence and/or severity of a disorder caused by stress-induced cellular damage in the middle or inner ear, such as vestibular disorders and hearing impairment involving middle or inner ear tissue, particularly middle or inner ear hair cells (e.g. hair-cell loss at the apical end of the cochlea), the stria vascularis (e.g. inflamed and damaged stria vascularis), and associated auditory nerves (neuronal signalling loss and apoptosis throughout the middle or inner ear).

The methods and uses described herein may be used to treat stress-induced cellular damage in the middle or inner ear which leads to cochlear hair cell loss and any disorder that arises as a consequence of hair cell loss in the ear, such as hearing impairment or deafness. The approach may be optimal for treatment of acute hearing loss shortly after the damage or stress has occurred.

The inner ear is a challenging area of the body to deliver pharmaceutical agents because many protective structures exist to protect the sensory cells and structures of the ear from damage caused by external agents.

The pharmaceutical formulations comprising a compound selected from an antioxidant, an anti-inflammatory agent, and an epithelial cell protective agent (e.g. probucol) are formulated to be compatible with the route of administration to the middle or inner ear. Suitable pharmaceutical formulations which may comprise a compound selected from an antioxidant, an anti-inflammatory agent, and an epithelial cell protective agent (e.g. probucol) are described below. The formulations may be delivered to the middle or inner ear by methods of administration known in the art. Suitable drug delivery systems which can deliver a compound selected from an antioxidant, an anti-inflammatory agent, and an epithelial cell protective agent (e.g. probucol) into the middle or inner ear are known in the art.

There are several ways that may be used to deliver a compound via local administration (intratympanical, round window, and cochleostomy). Cochleostomy approach is typically the best option for direct high efficacy of delivery, but it is invasive. The RWM is a semipermeable membrane with three layers which the outer epithelial layer contains some microvilli and abundant mitochondria, and inner layer has areas of discontinuous basement membrane that may provide space for substances to traverse the membrane. Nanoparticulate delivery systems ranging from silica-based materials to liposomes and nanogels represent an additional approach to otic formulation of drugs. Poloxamer-based delivery systems may also be used, such as OTIVIDEX (OTO-104, a micronized dexamethasone loaded P407 hydrogel formulation); OTO-311 (a P407 based formulation of the non-competitive N-methyl-D-aspartate (NMDA) receptor antagonist gacyclidine being developed for the treatment of tinnitus); and the poloxamer-based hydrogel formulation FX-322, a combinational therapy dubbed PCA (Progenitor Cell Activation).

In some embodiments, the pharmaceutical formulation is administered, or is formulated to be administered, to the outer, middle or inner ear by a method described below:
1) Application of the formulation, e.g. ear drops, to the outer ear such that the formulation moves through the ear canal to drain into the middle ear and subsequently into the inner ear;
2) Transtympanic (TT) syringe delivery of the pharmaceutical formulation to the middle ear as a bolus (0.1 to 1 mL);
3) TT syringe delivery of the pharmaceutical formulation specifically to the round window (RW) as a small gel or capsule (e.g. about 0.05 to 1 mL), e.g. a single transtympanic dose injected to the round window, or multiple transtympanic doses injected to the round window;
4) TT syringe delivery of the pharmaceutical formulation specifically to the oval window as a gel (e.g. about 0.05 to 1 mL);
5) TT delivery of the pharmaceutical formulation to the round window via a microwick or other catheter device;
6) TT and trans-RW delivery of the pharmaceutical formulation directly into the inner ear fluids via a needle;
7) TT delivery of the pharmaceutical formulation via a defect in the tympanic membrane (such as a grommet or perforation) with the pharmaceutical formulation delivered to the ear canal;
8) Transmastoid or transantral delivery of the pharmaceutical formulation which then drains into the middle ear and subsequently into the inner ear;
9) Direct delivery into the inner ear fluids, with the pharmaceutical formulation released from a ‘tube’, which is inserted into the cochlea;
10) Systemic delivery (coupled with methods of enhancing inner ear uptake) either via an intravenous, intramuscular, oral route or a combination of these;
11) Direct injection into the inner ear via the round window or cochleostomy with or without a catheter or device (such as a cochlear implant);
12) Direct injection into the inner ear via the inner ear appendages such as injection into the endolymphatic sac, semicircular canal, or vestibule with or without a catheter or device;
13) Direct injection into the inner ear via the oval window such as during the stapedotomy or stapedectomy, or via a middle ear prosthesis (such as a stapes prosthesis, or middle ear implant).

The above-described methods are known in the art. For example, drug delivery systems are described in the literature, e.g. M. Peppi, A. Marie, C. Belline & J. T. Borenstein (2018), “Intracochlear drug delivery systems: a novel approach whose time has come”, Expert Opinion on Drug Delivery, 15:4, 319-324; J. Wang and J-L. Puel, “Presbycusis: An Update on Cochlear Mechanisms and Therapies”, J. Clin. Med. 2020, 9, 218; J. Patel, M. Szczupak, S. Rajguru, C. Balaban and M. E. Hoffer (2019), “Inner Ear Therapeutics: An Overview of Middle Ear Delivery”, Front. Cell. Neurosci. 13:261; S. Nyberg, N. J. Abbott, X. Shi, P. S. Steyger, A. Dabdoub, “Delivery of therapeutics to the inner ear: The challenge of the blood-labyrinth barrier” Sci. Transl. Med. 11, eaao0935 (2019); A. A. McCall, E. E. Leary Swan, J. T. Borenstein, W. F. Sewell, S. G. Kujawa, and M. J. McKenna, “Drug Delivery for Treatment of Inner Ear Disease: Current State of Knowledge”, Ear Hear., 2010 April; 31(2): 156-165; K. Mäder, E. Lehner, A. Liebau, S. K. Plontke, “Controlled drug release to the inner ear: Concepts, materials, mechanisms, and performance”, Hearing Research, 368 (2018) 49-66; Yutian Ma, Andrew K. Wise, Robert K. Shepherd, and Rachael T. Richardson, “New molecular therapies for the treatment of hearing loss”, Pharmacology & Therapeutics 200 (2019), 190-209; (each of these documents is incorporated herein by reference).

In some embodiments, any two or more of the above-described methods to the middle or inner ear may be employed to administer the pharmaceutical formulation.

In some embodiments, the pharmaceutical formulation is administered by inserting a foam-like formulation into the ear, and the eardrum gently peeled off to allow entry of the formulation, and then sealed back.

In some embodiments, the pharmaceutical formulation is a solid or semi-solid formulation. In some embodiments, the pharmaceutical formulation is in the form of a liquid, mixture, suspension, emulsion, gel, foam or solution.

In some embodiments, the pharmaceutical formulation is administered by one of the modes of delivery listed below:
incorporated into or used in conjunction with tympanic membrane graft material as part of tympanoplasty or myringoplasty surgery;
incorporated into middle ear prostheses as part of ossicular chain reconstructive surgery;
used with established middle ear packing material such as Gelfoam or Gelfilm;
used as part of irrigation solution during middle ear or inner ear surgical procedures;
incorporated with suture material;
incorporated into ventilation tubes (grommets);
incorporated into bone condition implants such as the BAHA, Bonebridge, Ossia or Ponto device; or
incorporated into microsurgical drills.

In some embodiments, the pharmaceutical formulation is administered into the middle ear by application of a liquid or gel formulation by bolus transtympanic injection using, for example, a microfluidic device such as a fine syringe in a volume range of millilitres or microlitres. In some embodiments, the volume administered for a liquid or gel formulation is about 0.05 mL to about 1 mL. For example, the volume administered may be about 0.1 mL to about 0.8 mL, about 0.2 mL to about 0.7 mL, about 0.3 mL to about 0.6 mL, or about 0.4 mL to about 0.5 mL.

In some embodiments, the pharmaceutical formulation is administered by application of a liquid or gel formulation directly onto the round window or oval window membrane(s).

Application to these membranes may be accomplished using methods known in the art, e.g., intratympanic injection of a liquid or gel formulation, e.g., using a microfluidic device such as a fine syringe in a volume range of millilitres or microlitres. In some embodiments, the volume administered for a liquid or gel formulation is about 0.05 mL to about 1 mL. For example, the volume administered may be about 0.1 mL to about 0.8 mL, about 0.2 mL to about 0.7 mL, about 0.3 mL to about 0.6 mL, or about 0.4 mL to about 0.5 mL.

In some embodiments, the pharmaceutical formulation is administered by application of a liquid formulation by a catheter or wick delivery system. Catheter or wick delivery systems are known in the art. For example, such systems are described in Silverstein, H., Thompson, J., Rosenberg, S. I., Brown, N., Light, J., 2004. “Silverstein MicroWick”, Otolaryngol. Clin. North Am., 37, 1019-1034, which is incorporated herein by reference.

In some embodiments, the pharmaceutical formulation is administered directly to the middle or inner ear by a drug delivery system comprising the drug embedded in a silicone carrier such as a cochlear implant or middle ear implant. Drug delivery systems with active ingredients embedded in a silicone carrier are known in the art. Such systems are discussed in Plontke S K, Gotze G, Rahne T, Liebau A. Intracochlear drug delivery in combination with cochlear implants: Current aspects. HNO. 2017; 65(Suppl 1):19-28, which is incorporated herein by reference. A specific example with animal data is discussed in Farhadi M, Jalessi M, Salehian P, et al. Dexamethasone eluting cochlear implant: Histological study in animal model. Cochlear Implants Int. 2013; 14(1):45-50, which is incorporated herein by reference. A more recent example with human subjects is discussed in Briggs R, O'Leary S, Birman C, et al. Comparison of electrode impedance measures between a dexamethasone-eluting and standard Cochlear™ Contour Advance® electrode in adult cochlear implant recipients. Hear Res. 2020; 390:107924, which is incorporated herein by reference.

In some embodiments, the pharmaceutical formulation is administered directly to the middle or inner ear by a drug delivery system comprising the drug given in a droplet form through a defect in the tympanic membrane including through a ventilation tube. Drug delivery systems through a tympanic membrane defect are known in the art.

The inventors found that the local probucol delivery approach using transtympanic administration to the middle or inner ear is particularly useful in the treatment of stress-induced cellular damage in the middle or inner ear, because it allows the probucol to reach adequate therapeutic levels in the middle or inner ear, particularly in the cochlear region, rapidly and at higher concentrations.

In particular, the experimental data reported herein show that transtympanic administration of the pharmaceutical formulation of the invention promotes probucol diffusion deeply into the inner ear.

Therefore, in preferred embodiments, the pharmaceutical formulation of the present invention is administered by the transtympanic route, e.g., using a microfluidic device such as a fine syringe in a volume range of millilitres or microlitres. In some embodiments, the volume administered for a liquid or gel formulation is about 0.05 mL to about 1 mL. For example, the volume administered may be about 0.1 mL to about 0.8 mL, about 0.2 mL to about 0.7 mL, about 0.3 mL to about 0.6 mL, or about 0.4 mL to about 0.5 mL.

In some embodiments, the pharmaceutical formulation comprising a compound selected from an antioxidant, an anti-inflammatory agent, and an epithelial cell protective agent, e.g. probucol, is administered before the subject has developed, or before the subject is at risk to develop, a disorder caused by stress-induced cellular damage in the middle or inner ear. In some embodiments, the pharmaceutical formulation is administered after the subject has developed the disorder caused by stress-induced cellular damage in the middle or inner ear, such as a vestibular disorder, hearing impairment, hair cell degeneration, hair cell death and/or a condition characterised by hair cell damage. In some embodiments, the pharmaceutical formulation is administered to the subject at the moderate-to-profound stage of hearing loss, e.g., as a single transtympanic dose injected to the round window, or multiple transtympanic doses injected to the round window.

In some embodiments, the compound selected from an antioxidant, an anti-inflammatory agent, and an epithelial cell protective agent is probucol. Thus, in various aspects, the present invention provides the following:
a method for preventing, reducing or treating the incidence and/or severity of a disorder caused by stress-induced cellular damage in the middle or inner ear of a subject, the method comprising administering to the subject an effective amount of probucol;
a method for preventing, reducing or treating the incidence and/or severity of a vestibular disorder and/or hearing impairment in a subject, the method comprising administering to the subject an effective amount of probucol;
a method for preventing, reducing or inhibiting hair cell degeneration or hair cell death in a subject, the method comprising administering to the subject an effective amount of probucol;
use of probucol in the manufacture of a medicament for preventing, reducing or treating the incidence and/or severity of a disorder caused by stress-induced cellular damage in the middle or inner ear of a subject;
use of probucol in the manufacture of a medicament for preventing, reducing or treating the incidence and/or severity of a vestibular disorder and/or hearing impairment in a subject;
use of probucol in the manufacture of a medicament for preventing, reducing or inhibiting hair cell degeneration or hair cell death in a subject;
use of probucol for preventing, reducing or treating the incidence and/or severity of a disorder caused by stress-induced cellular damage in the middle or inner ear of a subject;
use of probucol for preventing, reducing or treating the incidence and/or severity of a vestibular disorder and/or hearing impairment in a subject;
use of probucol for preventing, reducing or inhibiting hair cell degeneration or hair cell death in a subject;
probucol for use in preventing, reducing or treating the incidence and/or severity of a disorder caused by stress-induced cellular damage in the middle or inner ear of a subject;
probucol for use in preventing, reducing or treating the incidence and/or severity of a vestibular disorder and/or hearing impairment in a subject;
probucol for use in preventing, reducing or inhibiting hair cell degeneration or hair cell death in a subject;
a pharmaceutical formulation comprising probucol as described herein for use in preventing, reducing or treating the incidence and/or severity of a disorder caused by stress-induced cellular damage in the middle or inner ear of a subject;
a pharmaceutical formulation comprising probucol as described herein for use in preventing, reducing or treating the incidence and/or severity of hearing impairment in a subject;
a pharmaceutical formulation comprising probucol as described herein for use in preventing, reducing or inhibiting hair cell degeneration or hair cell death in a subject.

Further diseases, disorders or conditions that may be related to, caused or characterised by stress-induced cellular damage in the middle or inner ear, such as hair cell degeneration and/or hair cell death, and that may be prevented or treated by the methods, uses and formulations of the present invention are, for example, Meniere's disease, cochlear hydrops, labyrinthitis, tinnitus, physical trauma (including surgical or implant trauma), vascular insults to the ear, complications from local or systemic infections (such as meningitis or otitis media), age-related hearing loss, acoustic trauma, ototoxicity, and autoimmune inner ear disorders. Thus, in some embodiments, the present invention provides a pharmaceutical formulation comprising a compound selected from an antioxidant, an anti-inflammatory agent, and an epithelial cell protective agent, e.g probucol, as described herein for use in a method of preventing or inhibiting middle or inner ear cell degeneration or cell death in a subject, wherein the cell degeneration or cell death is related to and/or caused by Meniere's disease, cochlear hydrops, labyrinthitis, tinnitus, physical trauma (including surgical or implant trauma), vascular insults to the ear, complications from local or systemic infections (such as meningitis or otitis media), age-related hearing loss (presbycusis), acoustic trauma, ototoxicity, and autoimmune inner ear disorders.

The subject may be a human subject, intended to comprise both adults and the “pediatric population” (where the term “pediatric population” is understood as the part of the population ranging from birth to eighteen years of age).

In some embodiments, the methods include steps of selecting a subject at risk of stress-induced cellular damage in the middle or inner ear, including a subject at risk of hair cell loss and/or a subject with hair cell loss. For example, any human experiencing or at risk for developing hair cell loss is a candidate for the treatment methods described herein. A human having or at risk for developing cochlear hair cell loss can hear less well than the average human being, or less well than a human before experiencing the hair cell loss. For example, hearing can be diminished by at least 5, 10, 30, 50% or more as determined by testing. Hearing loss can be impairment of pure tones or speech discrimination or both.

The stress-induced cellular damage in the middle or inner ear may be caused by, or the result of, any reason or type of event. For example, the subject may have hearing loss associated with cochlear hair cell loss for any reason, or as a result of any type of event. For example, a subject may be deaf or hard-of-hearing as a result of a physical ototoxic insult, e.g., a traumatic event, such as a physical trauma to a structure of the ear. In preferred embodiments, the subject may have (or be at risk of developing) hearing loss as result of exposure to a sudden loud noise, or a prolonged exposure to loud noises. For example, prolonged or repeated exposures to concert venues, airport runways, and construction areas can cause middle or inner ear damage and subsequent hearing loss; subjects who are subjected to high levels of environmental noise, e.g., in the home or workplace, may be treated using the methods, uses and formulations described herein. A subject can have a hearing disorder that results from aging, e.g., presbycusis, which is generally associated with normal aging processes, and can occur in subjects as young as 18, but is generally more marked in older subjects, e.g., subjects over age 40, 45, 50, 55, 60, 65, 70, 75, 80, 85, or 90. A subject can have tinnitus (characterised by ringing in the ears) due to loss of hair cells. A subject can experience a chemical ototoxic insult which induces cellular damage, wherein ototoxins include therapeutic drugs including antineoplastic agents, salicylates, quinines, and aminoglycoside antibiotics, e.g., as described further below, contaminants in foods or medicines, and environmental or industrial pollutants.

In some embodiments, the methods include administering a compound selected from an antioxidant, an anti-inflammatory agent, and an epithelial cell protective agent, e.g. probucol, to the subject within one, two, three, four, five, six, or seven days, or one, two, three, four, five, or six weeks of exposure to an ototoxic insult, e.g., a physical (noise, trauma) or chemical (ototoxin) insult that results in or could result in a loss of hair cells.

The compound selected from an antioxidant, an anti-inflammatory agent, and an epithelial cell protective agent, e.g. probucol, may be administered to a subject from one or more times per day to one or more times per week; including once every other day. The compound selected from an antioxidant, an anti-inflammatory agent, and an epithelial cell protective agent, e.g. probucol, may be administered as a sustained-release formulation. The skilled person will appreciate that certain factors may influence the dosage and timing required to effectively treat a subject, including but not limited to the severity of the disease or disorder, previous treatments, the general health and/or age of the subject, and other diseases present.

Moreover, treatment of a subject with a therapeutically effective amount of the compound selected from an antioxidant, an anti-inflammatory agent, and an epithelial cell protective agent, e.g. probucol, can include a single treatment or a series of treatments. In some embodiments, e.g., in subjects exposed to prolonged or repeated exposures to noise, e.g., normal noises such as are associated with activities of daily life (such as lawnmowers, trucks, motorcycles, airplanes, music (e.g., from personal listening devices), sporting events, etc.), or loud noises, e.g., at concert venues, airports, and construction areas, that can cause middle or inner ear damage and subsequent hearing loss; e.g., subjects who are subjected to high levels of environmental noise, e.g., in the home or workplace, may be treated with repeated, e.g., periodic, doses of the pharmaceutical formulations, e.g., to prevent (reduce the risk of) or delay progression of hearing loss.

In some embodiments, the pharmaceutical formulation comprising a compound selected from an antioxidant, an anti-inflammatory agent, and an epithelial cell protective agent, e.g. probucol, is in the form of a solution, a mixture, a suspension, microparticles, nanoparticles (e.g. encapsulated nanoparticles), or a gel.

In some embodiments, the pharmaceutical formulation is in the form of a nanocapsule gel, which may, for example, be injected on the round window, or be used to coat a catheter inserted onto the cochlea (e.g. with cochlear implants). The inventors believe that the nanocapsule gel will facilitate round-window permeation by the compound (e.g. probucol), control compound diffusion and specific cell targeting (using cell-specific antibodies on the surface of compound-nanocapsules) once the nanocapsules are within the cochlea. Upon cellular contact, the antibody will trigger the endocytosis of the nanocapsules and the compound (e.g. probucol) will be released within the targeted cells. The nanocapsules then safely dissolve away.

Nanocapsules are known in the art and can be tailored to suit various demands of drug delivery. Nanocapsules are, for example, described in J. Y. Yoon, K. J. Yang, S. N. Park, D. K. Kim, J. D. Kim, “The effect of dexamethasone/cell-penetrating peptide nanoparticles on gene delivery for inner ear therapy”, Int J Nanomedicine 11 (2016) 6123-6134.

When the pharmaceutical formulation is in form of a solution, the concentration of the compound selected from an antioxidant, an anti-inflammatory agent, and an epithelial cell protective agent, e.g. probucol, may, for example, be from 0.1 to 1000 mg/mL, e.g. from 0.1 to 2 mg/mL, from 0.2 to 5 mg/mL, from 1 to 5 mg/mL, from 2 to 8 mg/mL, from 5 to 10 mg/mL, from 10 to 20 mg/mL, from 20 to 30 mg/mL, from 30 to 40 mg/mL, from 40 to 50 mg/mL, from 50 to 60 mg/mL, from 60 to 70 mg/mL, from 70 to 80 mg/mL, from 80 to 90 mg/mL, from 90 to 100 mg/mL, from 10 to 100 mg/mL, from 100 to 200 mg/mL, from 200 to 300 mg/mL, from 300 to 400 mg/mL, from 500 to 1000 mg/mL, from 400 to 500 mg/mL, from 500 to 600 mg/mL, from 600 to 700 mg/mL, from 700 to 800 mg/mL, from 800 to 900 mg/mL, or from 900 to 1000 mg/mL of the compound selected from an antioxidant, an anti-inflammatory agent, and an epithelial cell protective agent, e.g. probucol.

When the pharmaceutical formulation is in form of a suspension, the concentration of the compound selected from an antioxidant, an anti-inflammatory agent, and an epithelial cell protective agent, e.g. probucol, may, for example, be from 0.1 to 1000 mg/mL, e.g. from 0.1 to 2 mg/mL, from 0.2 to 5 mg/mL, from 1 to 5 mg/mL, from 2 to 8 mg/mL, from 5 to 10 mg/mL, from 10 to 20 mg/mL, from 20 to 30 mg/mL, from 30 to 40 mg/mL, from 40 to 50 mg/mL, from 50 to 60 mg/mL, from 60 to 70 mg/mL, from 70 to 80 mg/mL, from 80 to 90 mg/mL, from 90 to 100 mg/mL, from 10 to 100 mg/mL, from 100 to 200 mg/mL, from 200 to 300 mg/mL, from 300 to 400 mg/mL, from 500 to 1000 mg/mL, from 400 to 500 mg/mL, from 500 to 600 mg/mL, from 600 to 700 mg/mL, from 700 to 800 mg/mL, from 800 to 900 mg/mL, or from 900 to 1000 mg/mL of the compound selected from an antioxidant, an anti-inflammatory agent, and an epithelial cell protective agent, e.g. probucol.

When the pharmaceutical formulation is in form of a microparticles, the concentration of the compound selected from an antioxidant, an anti-inflammatory agent, and an epithelial cell protective agent, e.g. probucol, may, for example, be from 0.1 to 1000 mg/mL, e.g. from 0.1 to 2 mg/mL, from 0.2 to 5 mg/mL, from 1 to 5 mg/mL, from 2 to 8 mg/mL, from 5 to 10 mg/mL, from 10 to 20 mg/mL, from 20 to 30 mg/mL, from 30 to 40 mg/mL, from 40 to 50 mg/mL, from 50 to 60 mg/mL, from 60 to 70 mg/mL, from 70 to 80 mg/mL, from 80 to 90 mg/mL, from 90 to 100 mg/mL, from 10 to 100 mg/mL, from 100 to 200 mg/mL, from 200 to 300 mg/mL, from 300 to 400 mg/mL, from 500 to 1000 mg/mL, from 400 to 500 mg/mL, from 500 to 600 mg/mL, from 600 to 700 mg/mL, from 700 to 800 mg/mL, from 800 to 900 mg/mL, or from 900 to 1000 mg/mL of the compound selected from an antioxidant, an anti-inflammatory agent, and an epithelial cell protective agent, e.g. probucol.

When the pharmaceutical formulation is in form of a nanoparticles, the concentration of the compound selected from an antioxidant, an anti-inflammatory agent, and an epithelial cell protective agent, e.g. probucol, may, for example, be from 0.1 to 1000 mg/mL, e.g. from 0.1 to 2 mg/mL, from 0.2 to 5 mg/mL, from 1 to 5 mg/mL, from 2 to 8 mg/mL, from 5 to 10 mg/mL, from 10 to 20 mg/mL, from 20 to 30 mg/mL, from 30 to 40 mg/mL, from 40 to 50 mg/mL, from 50 to 60 mg/mL, from 60 to 70 mg/mL, from 70 to 80 mg/mL, from 80 to 90 mg/mL, from 90 to 100 mg/mL, from 10 to 100 mg/mL, from 100 to 200 mg/mL, from 200 to 300 mg/mL, from 300 to 400 mg/mL, from 500 to 1000 mg/mL, from 400 to 500 mg/mL, from 500 to 600 mg/mL, from 600 to 700 mg/mL, from 700 to 800 mg/mL, from 800 to 900 mg/mL, or from 900 to 1000 mg/mL of the compound selected from an antioxidant, an anti-inflammatory agent, and an epithelial cell protective agent, e.g. probucol.

When the pharmaceutical formulation is in form of a gel, the concentration of the compound selected from an antioxidant, an anti-inflammatory agent, and an epithelial cell protective agent, e.g. probucol, may, for example, be from 0.1 to 1000 mg/mL, e.g. from 0.1 to 2 mg/mL, from 0.2 to 5 mg/mL, from 1 to 5 mg/mL, from 2 to 8 mg/mL, from 5 to 10 mg/mL, from 10 to 20 mg/mL, from 20 to 30 mg/mL, from 30 to 40 mg/mL, from 40 to 50 mg/mL, from 50 to 60 mg/mL, from 60 to 70 mg/mL, from 70 to 80 mg/mL, from 80 to 90 mg/mL, from 90 to 100 mg/mL, from 10 to 100 mg/mL, from 100 to 200 mg/mL, from 200 to 300 mg/mL, from 300 to 400 mg/mL, from 500 to 1000 mg/mL, from 400 to 500 mg/mL, from 500 to 600 mg/mL, from 600 to 700 mg/mL, from 700 to 800 mg/mL, from 800 to 900 mg/mL, or from 900 to 1000 mg/mL of the compound selected from an antioxidant, an anti-inflammatory agent, and an epithelial cell protective agent, e.g. probucol.

The pharmaceutical formulations comprising the compound selected from an antioxidant, an anti-inflammatory agent, and an epithelial cell protective agent, e.g. probucol, are formulated to be compatible with the intended route of administration.

In one embodiment, the pharmaceutical formulation comprises a cyclodextrin (e.g. (2-hydroxypropyl)-β-cyclodextrin), a bile acid or bile salt (e.g. deoxycholic acid, cholic acid, taurocholic acid, glycocholic acid, glycodeoxycholic acid, taurodeoxycholic acid, ursodeoxycholic acid, taurochenodeoxycholic acid, lithocholic acid, glycolithocholic acid, chenodeoxycholic acid, taurolithocholic acid, tauroursodeoxycholic acid, obeticholic acid, any muricholic acid (e.g. α-muricholic acid, β-muricholic acid, γ-muricholic acid, ω-muricholic acid), glycomuricholic acid, tauromuricholic acid, or glycochenodeoxycholic acid, or a salt thereof), and the compound selected from an antioxidant, an anti-inflammatory agent, and an epithelial cell protective agent, e.g. probucol, in a form suitable for cochlea (inner ear) delivery.

In one embodiment, the pharmaceutical formulation comprises a cyclodextrin (e.g. (2-hydroxypropyl)-β-cyclodextrin), a bile acid or bile salt (e.g. deoxycholic acid, cholic acid, taurocholic acid, glycocholic acid, glycodeoxycholic acid, taurodeoxycholic acid, ursodeoxycholic acid, taurochenodeoxycholic acid, lithocholic acid, glycolithocholic acid, chenodeoxycholic acid, taurolithocholic acid, tauroursodeoxycholic acid, obeticholic acid, any muricholic acid (e.g. α-muricholic acid, β-muricholic acid, γ-muricholic acid, ω-muricholic acid), glycomuricholic acid, tauromuricholic acid, or glycochenodeoxycholic acid, or a salt thereof), and the compound selected from an antioxidant, an anti-inflammatory agent, and an epithelial cell protective agent, e.g. probucol, in a gel formulation suitable for cochlea (inner ear) delivery.

In one embodiment, the pharmaceutical formulation comprises (2-hydroxypropyl)-β-cyclodextrin, deoxycholic acid and/or ursodeoxycholic acid, and probucol in a gel formulation suitable for cochlea (inner ear) probucol delivery.

In one embodiment, the pharmaceutical formulation comprises (2-hydroxypropyl)-β-cyclodextrin, deoxycholic acid, ursodeoxycholic acid, and probucol in a gel formulation suitable for cochlea (inner ear) probucol delivery.

In one embodiment, the pharmaceutical formulation comprises a cyclodextrin, deoxycholic acid, and probucol nanoparticles in an ultrasonic gel, for cochlea (inner ear) probucol delivery.

In one embodiment, the pharmaceutical formulation comprises a cyclodextrin, deoxycholic acid, ursodeoxycholic acid, and probucol nanoparticles in an ultrasonic gel, for cochlea (inner ear) probucol delivery.

In one embodiment, the pharmaceutical formulation comprises a (2-hydroxypropyl)-β-cyclodextrin, deoxycholic acid, ursodeoxycholic acid, and probucol nanoparticles in an ultrasonic gel, for cochlea (inner ear) probucol delivery.

In some embodiments, a pharmaceutical formulation in the form of a gel may include the following components: probucol, one or more bile acids, one or more cyclodextrins, polysorbate 80 (Tween 80), glycerol, propylene glycol, a water-soluble gel, and water.

For example, the pharmaceutical formulation may include the following components:

| Component | Amount |
|---|---|
| Water | about 200-500 mL |
| (2-Hydroxypropyl)-β-cyclodextrin | about 1-15 g |
| Polysorbate 80 (Tween 80) | about 1-10 mL |
| Glycerol | about 1-10 mL |
| Propylene glycol | about 1-20 mL |
| Metron Water Soluble Gel | about 1-20 g |
| Probucol | about 0.1-10 g |
| Deoxycholic acid (DCA) | about 0.1-10 g |
| Ursodeoxycholic acid (UDCA) | about 0.1-10 g |

Two specific formulation examples are provided below:

| Component | Amount | Amount |
|---|---|---|
| Water | about 350 mL | about 350 mL |
| (2-Hydroxypropyl)-β-cyclodextrin | about 7 g | about 7 g |
| Polysorbate 80 (Tween 80) | about 5 mL | about 5 mL |
| Glycerol | about 6 mL | about 5 mL |
| Propylene glycol | about 12 mL | about 11 mL |
| Metron Water Soluble Gel | about 6.5 g | about 6 g |
| Probucol | about 4 g | about 4 g |
| Deoxycholic acid (DCA) | about 3 g | about 2 g |
| Ursodeoxycholic acid (UDCA) | about 1 g | about 3 g |

A typical method of preparation is as follows. A mixture of (2-hydroxypropyl)-β-cyclodextrin in water is mixed with stirring at about 80° C. for about 1 hour. Probucol is then added and the resulting mixture is heated with stirring to about 80° C. for about 1 hour. Tween 80 is then added, and the resulting mixture is heated with stirring to about 80° C. for about 1 hour. Ursodeoxycholic acid (UDCA) is then added, and the resulting mixture is heated with stirring to about 80° C. for about 1 hour. Deoxycholic acid is then added, and the resulting mixture is heated with stirring to about 80° C. for about 1 hour. Metron Water Soluble Gel is then added, and the resulting mixture is heated with stirring to about 80° C. for about 1 hour. Glycerol and propylene glycol are then added, and the resulting mixture is heated with stirring to about 80° C. for about 1 hour. The resulting mixture is then stirred at about 80° C. for a further hour, and then water is added to make up the volume to the desired amount.

Processes for formulating suitable pharmaceutical formulations are known in the art, see, e.g., Remington: The Science and Practice of Pharmacy. 21st ed., 2005; and the books in the series Drugs and the Pharmaceutical Sciences: A Series of Textbooks and Monographs (Dekker, NY). For example, solutions or suspensions can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The pH may be adjusted with acids or bases, such as hydrochloric acid or sodium hydroxide. The parenteral preparation may be enclosed in ampoules, disposable syringes or multiple dose vials made of glass or plastic.

Pharmaceutical formulations suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the formulation must be sterile and should be fluid to the extent that easy syringability exists. It should be stable under the conditions of manufacture and storage and must be preserved against the contaminating action of microorganisms such as bacteria and fungi. The carrier may be a solvent or dispersion medium containing, for example, water, ethanol, polyol (for example, glycerol, propylene glycol, and liquid polyetheylene glycol, and the like), and suitable mixtures thereof. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the formulation. Prolonged absorption of the injectable formulations may be brought about by including in the formulation an agent that delays absorption, for example, aluminium monostearate and gelatin.

Sterile injectable solutions may be prepared by incorporating the active ingredient (e.g. probucol) in the required amount in an appropriate solvent with one or a combination of ingredients enumerated above, as required, followed by filtered sterilisation. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

The pharmaceutical formulations may be included in a container, pack, or dispenser together with instructions for administration.

In a further embodiment, the pharmaceutical formulation comprising the compound selected from an antioxidant, an anti-inflammatory agent, and an epithelial cell protective agent, e.g. probucol, may comprise at least one further active ingredient. The further active ingredient may be an antibacterial agent, an antiviral agent, an antifungal agent, an anti-inflammatory agent, an osmotically active substance (e.g. mannitol), or other suitable therapeutically or pharmacologically active agent. In some embodiments, the further active ingredient is a steroid, for example, selected from dexamethasone, methylprednisolone and prednisolone. In some embodiments, the further active ingredient is an antibiotic, such as gentamicin. In some embodiments the further active ingredient may have otoprotective effects, specific to preventing middle or inner ear damage related to chemotherapy, such as that which occurs with cisplatin treatment or radiotherapy.

In a further embodiment, the pharmaceutical formulation comprising the compound selected from an antioxidant, an anti-inflammatory agent, and an epithelial cell protective agent, e.g. probucol, may be administered simultaneously, separately or sequentially in combination with at least one further active ingredient, also following different routes of administration for each active ingredient. The further active ingredient may be an antibacterial agent, an antiviral agent, an antifungal agent, an anti-inflammatory agent, a chemotherapeutic agent, an osmotically active substance (e.g. mannitol), or other suitable therapeutically or pharmacologically active agent. In particular, the further active ingredient may be an ototoxic or oto-irritant therapeutic drug; commonly used ototoxic drugs include aminoglycosides, platinum-based chemotherapeutic agents, loop diuretics, macrolide antibiotics, and antimalarials. In some embodiments, the further active ingredient is a steroid, for example, selected from dexamethasone, methylprednisolone and prednisolone.

In some embodiments, the pharmaceutical formulation comprising the compound selected from an antioxidant, an anti-inflammatory agent, and an epithelial cell protective agent, e.g. probucol, may be administered to a subject being treated with gene therapy (e.g. viral vectors), or stem cell therapy.

Where appropriate, following treatment, the subject may be tested for an improvement in, for example, hearing or other symptoms related to middle or inner ear disorders. Methods for measuring hearing are well-known and include pure tone audiometry, air conduction, and bone conduction tests. These exams measure the limits of loudness (intensity) and pitch (frequency) that a subject can hear. Hearing tests in humans include behavioural observation audiometry (for infants to seven months), visual reinforcement orientation audiometry (for children 7 months to 3 years); play audiometry for children older than 3 years; and standard audiometric tests for older children and adults, e.g., whispered speech, pure tone audiometry; tuning fork tests; brain stem auditory evoked response (BAER) testing or auditory brain stem evoked potential (ABEP) testing. Oto-acoustic emission testing may be used to test the functioning of the cochlear hair cells, and electro-cochleography provides information about the functioning of the cochlea and the first part of the nerve pathway to the brain. In some embodiments, treatment can be continued with or without modification or can be stopped.

In some embodiments, the pharmaceutical formulation may comprise one or more components of the formulation described below in Section 2.2, or be administered by a mode of administration described below in Section 2.2.

### 2.2. Pharmaceutical Formulations of the Present Invention

In a further aspect, the present invention provides a pharmaceutical formulation that comprises an active ingredient and an agent that enhances the bioavailability of the active ingredient. Advantageously, the pharmaceutical formulation of this aspect of the invention can be employed where it is desired to enhance or increase the bioavailability of an active ingredient, e.g. in instances where an active ingredient is highly lipophilic and/or poorly water soluble (e.g. probucol).

In one aspect, the present invention provides a pharmaceutical formulation comprising:
(i) an active ingredient;
(ii) an agent that enhances the bioavailability of the active ingredient, wherein the agent comprises an amphiphilic compound of the formula (I):

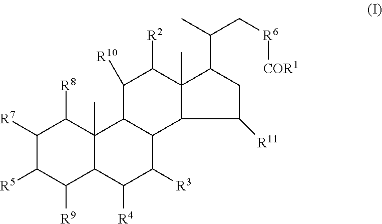

wherein:
   each R¹, R², R³, R⁴, R⁵, R⁷, R⁸, R⁹, R¹⁰ and R¹¹ is independently selected from H, substituted or unsubstituted C₁₋₃₀ alkyl, substituted or unsubstituted C₂₋₃₀ alkenyl, substituted or unsubstituted C₂₋₃₀ alkynyl, substituted or unsubstituted C₃₋₃₀ cycloalkyl, substituted or unsubstituted aryl, substituted or unsubstituted arylalkyl, substituted or unsubstituted heteroaryl, or GL; wherein when R¹, R², R³, R⁴, R⁵, R⁷, R⁸, R⁹, R¹⁰ or R¹¹ is substituted, the substituent is independently selected from OH, F, SH, ═O, ═S, Cl, Br, SC₁₋₆ alkyl, C₁₋₆ alkyl or C₁₋₆ alkoxy;
   G is absent or is selected from O, S, PL, CL₂, or NL;
   each L is independently selected from H, a metallic ion, substituted or unsubstituted C₁₋₃₀ alkyl, substituted or unsubstituted C₂₋₃₀ alkenyl, substituted or unsubstituted C₂₋₃₀ alkynyl, substituted or unsubstituted C₃₋₃₀ cycloalkyl, a substituted or unsubstituted benzyl radical, —CH₂CO₂H, or —(CH₂)₂SO₃H; wherein when L is substituted, the substituent is independently selected from OH, SH, ═O, ═S, F, Cl, Br, SC₁₋₆ alkyl, C₁₋₆ alkyl or C₁₋₆ alkoxy; or when L is bonded to R¹, L may be an amino acid; and
   R⁶ is —(CH₂)ₙ— wherein n is 0 to 12;
   or a stereoisomer thereof, or a pharmaceutically acceptable salt thereof; and
(iii) optionally a pharmaceutically acceptable carrier;

wherein the formulation is formulated to be administered to the middle or inner ear.

Without wishing to be bound by theory, the inventors believe that the steroidal backbone of formula (I) conserves the lipophilicity and hence leads to permeation-enhancing effects.

Advantageously, increased lipophilicity and hence increased permeation enhancement occurs when there are fewer freely available H atoms, a higher number of methyl groups and no amino acid (e.g. taurine or glycine) conjugation to the carboxylic acid group with the R¹ group being a pivotal site for the molecule's ability to act as a lipophilic surfactant. The inventors believe that the main cause of permeation enhancing is the ability to have sufficient surface charge to interact with the epithelial cell's surface, and enable surfactant effects by having a water-soluble moiety to mix with the body's biological fluids and a lipid-soluble moiety to mix with the cell lipid membrane. Among the natural bile acids, deoxycholic acid (DCA) and lithocholic acid (LCA) have the structural elements that enable them to be particularly good permeation enhancers.

In some embodiments, the agent that enhances the bioavailability of the active ingredient comprises nanoparticles or microparticles of the amphiphilic compound of the formula (I) or a stereoisomer thereof, or a pharmaceutically acceptable salt thereof. Suitable particle sizes may be obtained by methods known in the art, for example, by micronisation.

In some embodiments, the agent that enhances the bioavailability of the active ingredient comprises the amphiphilic compound of the formula (I) or a stereoisomer thereof, or a pharmaceutically acceptable salt thereof, is formulated into a gel or liquid comprising the amphiphilic compound of the formula (I) or a stereoisomer thereof, or a pharmaceutically acceptable salt thereof.

In some embodiments, the pharmaceutical formulation is in the form of a solution, a mixture, a suspension, microparticles, nanoparticles (e.g. encapsulated nanoparticles), or a gel.

In some embodiments, the pharmaceutical formulation is in the form of a nanocapsule gel, which may, for example, be injected on the round window, or be used to coat a catheter inserted onto the cochlea (e.g. with cochlear implants). The inventors believe that the nanocapsule gel will facilitate round-window permeation of the active ingredient (such as probucol), control probucol diffusion and specific cell targeting (using cell-specific antibodies on the surface of active ingredient containing-nanocapsules) once the nanocapsules are within the cochlea. Upon cellular contact, the antibody will trigger the endocytosis of the nanocapsules and active ingredient (such as probucol) will be released within the targeted cells.

Nanocapsules are known in the art and can be tailored to suit various demands of drug delivery. Nanocapsules are, for example, described in J. Y. Yoon, K. J. Yang, S. N. Park, D. K. Kim, J. D. Kim, “The effect of dexamethasone/cell-penetrating peptide nanoparticles on gene delivery for inner ear therapy”, Int J Nanomedicine 11 (2016) 6123-6134.

In another aspect, the present invention provides a pharmaceutical formulation comprising:
(i) an active ingredient;
(ii) an agent that enhances the bioavailability of the active ingredient, wherein the agent comprises an amphiphilic compound selected from deoxycholic acid, cholic acid, taurocholic acid, glycocholic acid, glycodeoxycholic acid, taurodeoxycholic acid, ursodeoxycholic acid, taurochenodeoxycholic acid, lithocholic acid, glycolithocholic acid, chenodeoxycholic acid, taurolithocholic acid, tauroursodeoxycholic acid, obeticholic acid, α-muricholic acid, R-muricholic acid, γ-muricholic acid, ω-muricholic acid, glycomuricholic acid, tauromuricholic acid, and glycochenodeoxycholic acid, or a pharmaceutically acceptable salt, derivative or metabolite thereof; and
(iii) optionally a pharmaceutically acceptable carrier;

wherein the formulation is formulated to be administered to the middle or inner ear.

### (i) Active Ingredient

The pharmaceutical formulation of this aspect of the invention may include any active ingredient where it is desired to enhance or increase the bioavailability of the active ingredient, e.g. in instances where the active ingredient is highly lipophilic and/or poorly water soluble. An example of a highly lipophilic active ingredient is probucol.

In some embodiments, the active ingredient is an active ingredient which is suitable for preventing, reducing or treating the incidence and/or severity of a disorder caused by stress-induced cellular damage in the middle or inner ear of a subject, such as vestibular disorders, hearing impairment, and conditions related to hair cell degeneration or hair cell death.

Active ingredients that may be usefully administered with the pharmaceutical formulation include, for example, corticosteroids, antibiotics, neurotrophins, growth factors, anti-fibrotic agents, and stem cells promoting agents.

Active ingredients that may be usefully administered with the pharmaceutical formulation also include, for example, from a compound selected from an antioxidant, an anti-inflammatory agent, and an epithelial cell protective agent.

Examples of active ingredients which may be included in the pharmaceutical formulation are: probucol, succinobucol (4-[2,6-ditert-butyl-4-[2-(3,5-ditert-butyl-4-hydroxyphenyl)sulfanylpropan-2-ylsulfanyl]phenoxy]-4-oxobutanoic acid), probucol analogues, statins, gliclazide, and thiol antioxidants.

Examples of antioxidants include vitamins, magnesium, Ebselen (SPI-1005), N-acetylcysteine, Coenzyme Q10, alpha-lipoic acid, sodium thiosulphate, Gingko biloba, and steroids.

Statins are a class of cholesterol-reducing drugs which are available commercially. Statins suitable for use in the invention include, for example, atorvastatin and simvastatin.

In some embodiments of a pharmaceutical formulation suitable for preventing, reducing or treating the incidence and/or severity of a disorder caused by stress-induced cellular damage in the middle or inner ear, the formulation comprises from 0.1 to 99% w/w of an active ingredient (e.g. from 0.1 to 50% w/w), preferably from 1 to 50% w/w of an active ingredient, more preferably from 5 to 50% w/w of an active ingredient. For example, the formulation may comprise about 0.1% w/w, about 0.2% w/w, about 0.3% w/w, about 0.4% w/w, about 0.5% w/w, about 0.6% w/w, about 0.7% w/w, about 0.8% w/w, about 0.9% w/w, about 1% w/w, about 2% w/w, about 3% w/w, about 4% w/w, about 5% w/w, about 6% w/w, about 7% w/w, about 8% w/w, about 9% w/w, about 10% w/w, about 11% w/w, about 12% w/w, about 13% w/w, about 14% w/w, about 15% w/w, about 16% w/w, about 17% w/w, about 18% w/w, about 19% w/w, about 20% w/w, about 25% w/w, about 30% w/w, about 35% w/w, about 40% w/w, about 45% w/w, about 50% w/w, about 55% w/w, about 60% w/w, about 65% w/w, about 70% w/w, about 75% w/w, about 80% w/w, about 85% w/w, about 90% w/w, or about 95% w/w of an active ingredient.

In some embodiments, the active ingredient is probucol. When the pharmaceutical formulation suitable for preventing, reducing or treating the incidence and/or severity of a disorder caused by stress-induced cellular damage in the middle or inner ear comprises probucol, the formulation comprises from 0.1 to 99% w/w of probucol (e.g. from 0.1 to 50% w/w), preferably from 1 to 50% w/w of probucol, more preferably from 5 to 50% w/w of probucol. For example, the formulation may comprise about 0.1% w/w, about 0.2% w/w, about 0.3% w/w, about 0.4% w/w, about 0.5% w/w, about 0.6% w/w, about 0.7% w/w, about 0.8% w/w, about 0.9% w/w, about 1% w/w, about 2% w/w, about 3% w/w, about 4% w/w, about 5% w/w, about 6% w/w, about 7% w/w, about 8% w/w, about 9% w/w, about 10% w/w, about 11% w/w, about 12% w/w, about 13% w/w, about 14% w/w, about 15% w/w, about 16% w/w, about 17% w/w, about 18% w/w, about 19% w/w, about 20% w/w, about 25% w/w, about 30% w/w, about 35% w/w, about 40% w/w, about 45% w/w, about 50% w/w, about 55% w/w, about 60% w/w, about 65% w/w, about 70% w/w, about 75% w/w, about 80% w/w, about 85% w/w, about 90% w/w, or about 95% w/w of probucol.

In some embodiments, the active ingredient is a steroid, such as selected from dexamethasone, methylprednisolone and prednisolone. When the pharmaceutical formulation suitable for preventing, reducing or treating the incidence and/or severity of a disorder caused by stress-induced cellular damage in the middle or inner ear comprises a steroid, the formulation comprises from 0.1 to 99% w/w of the steroid (e.g. from 0.1 to 50% w/w), preferably from 1 to 50% w/w of the steroid, more preferably from 5 to 50% w/w of the steroid.

(ii) Agent that Enhances the Bioavailability of the Active Ingredient

The agent that enhances the bioavailability of the active ingredient comprises an amphiphilic compound of the formula (I)

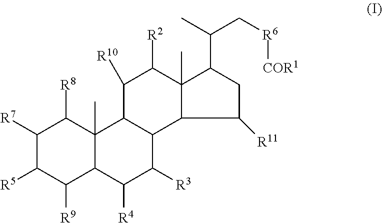

wherein:
each R¹, R², R³, R⁴, R⁵, R⁷, R⁸, R⁹, R¹⁰ and R¹¹ is independently selected from H, substituted or unsubstituted C₁₋₃₀ alkyl, substituted or unsubstituted C₂₋₃₀ alkenyl, substituted or unsubstituted C₂₋₃₀ alkynyl, substituted or unsubstituted C₃₋₃₀ cycloalkyl, substituted or unsubstituted aryl, substituted or unsubstituted heteroaryl, or GL; wherein when R¹, R², R³, R⁴, R⁵, R⁷, R⁸, R⁹, R¹⁰ or R¹¹ is substituted, the substituent is independently selected from OH, F, SH, ═O, ═S, Cl, Br, SC₁₋₆ alkyl, C₁₋₆ alkyl or C₁₋₆ alkoxy;
G is absent or is selected from O, S, PL, CL₂, or NL;
each L is independently selected from H, a metallic ion, substituted or unsubstituted C₁₋₃₀ alkyl, substituted or unsubstituted C₂₋₃₀ alkenyl, substituted or unsubstituted C₂₋₃₀ alkynyl, substituted or unsubstituted C₃₋₃₀ cycloalkyl, a substituted or unsubstituted benzyl radical, —CH₂CO₂H, or —(CH₂)₂SO₃H; wherein when L is substituted, the substituent is independently selected from OH, F, Cl, SH, ═O, ═S, Br, SC₁₋₆ alkyl, C₁₋₆ alkyl or C₁₋₆ alkoxy; or when L is bonded to R¹, L may be an amino acid; and
R⁶ is —(CH₂)ₙ— wherein n is 0 to 12;
or a stereoisomer thereof, or a pharmaceutically acceptable salt thereof.

In some embodiments, each R¹, R², R³, R⁴, R⁵, R⁷, R⁸, R⁹, R¹⁰ and R¹¹ is independently selected from H, substituted or unsubstituted C₁₋₁₂ alkyl (e.g. substituted or unsubstituted C₁₋₆ alkyl or substituted or unsubstituted C₁₋₄ alkyl); substituted or unsubstituted C₂₋₁₂ alkenyl (e.g. substituted or unsubstituted C₂₋₆ alkenyl or substituted or unsubstituted C₂₋₄ alkenyl); substituted or unsubstituted C₂₋₁₂ alkynyl (e.g. substituted or unsubstituted C₂₋₆ alkynyl or substituted or unsubstituted C₂₋₄ alkynyl); substituted or unsubstituted C₃₋₈ cycloalkyl; substituted or unsubstituted Ce aryl (e.g. substituted or unsubstituted phenyl); or substituted or unsubstituted heteroaryl (e.g. substituted or unsubstituted pyridyl). In some embodiments, when R¹, R², R³, R⁴, R⁵, R⁷, R⁸, R⁹, R¹⁰ or R¹¹ is substituted, the substituent is independently selected from F, Cl, OH, SH, Br, SC₁₋₄ alkyl, C₁₋₄ alkyl or C₁₋₄ alkoxy. In some embodiments, when R¹, R², R³, R⁴, R⁵, R⁷, R⁸, R⁹, R¹⁰ or R¹¹ is substituted, it is substituted with 1, 2, 3 or 4 substituents independently selected from F, Cl, Br, OH, SH, ═O, ═S, SC₁₋₄ alkyl, C₁₋₄ alkyl or C₁₋₄ alkoxy.

In some embodiments, G is absent. In some embodiments, G is selected from O, S, PL, CL₂, or NL.

In some embodiments, L is H; substituted or unsubstituted C₁₋₁₂ alkyl (e.g. substituted or unsubstituted C₁₋₆ alkyl or substituted or unsubstituted C₁₋₄ alkyl); substituted or unsubstituted C₂₋₁₂ alkenyl (e.g. substituted or unsubstituted C₂₋₆ alkenyl or substituted or unsubstituted C₂₋₄ alkenyl); substituted or unsubstituted C₂₋₁₂ alkynyl (e.g. substituted or unsubstituted C₂₋₆ alkynyl or substituted or unsubstituted C₂₋₄ alkynyl); substituted or unsubstituted C₃₋₈ cycloalkyl; a substituted or unsubstituted benzyl radical, —CH₂CO₂H, or —(CH₂)₂SO₃H. In some embodiments, when L is substituted, the substituent is independently selected from F, Cl, OH, SH, ═O, ═S, Br, SC₁₋₄ alkyl, C₁₋₄ alkyl or C₁₋₄ alkoxy. In some embodiments, when L is substituted, it is substituted with 1, 2, 3 or 4 substituents independently selected from F, Cl, OH, SH, ═O, ═S, Br, SC₁₋₄ alkyl, C₁₋₄ alkyl or C₁₋₄ alkoxy.

In some embodiments, L is a metallic ion selected from, for example, metal(I) ions (e.g. Na⁺, K⁺, and Li⁺), and metal(II) ions (e.g. cadmium(II) ion, iron(II) ion, lead(II) ion, zinc(II) ion, copper(II) ion, magnesium(II) ion, and manganese(II) ion). Thus in some embodiments, a metallic cation (e.g. Na⁺, K⁺, Li⁺, Cd²⁺, Fe²⁺, Pb²⁺, Zn²⁺, Cu²⁺, Mg²⁺, or Mn²⁺) is bonded to an organic moiety.

In some embodiments, L is bonded to R¹, and L is an amino acid. The amino acid may be any amino acid, such as, for example, any of the available main types of amino acids (20 in total). For example, the amino acid may be selected from alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine, and taurine.

In some embodiments, the amino acid is glycine, taurine or alanine. In some embodiments, the amino acid is glycine. In some embodiments, the amino acid is taurine.

In some embodiments, n is 0. In some embodiments, n is 1, 2, 3, 4, 5, 6, 7, 8, 9, 10, 11 or 12. In some embodiments, n is 1, 2, 3, 4 or 5.

In some embodiments, the amphiphilic compound of the formula (I) has two hydroxyl (—OH) groups attached to two different rings of formula (I), e.g. two different rings of a carboxylated steroidal compound of formula (I).

In some embodiments, the amphiphilic compound of the formula (I) is present in the pharmaceutical formulation in an amount of from about 0.1% w/w to about 10% w/w, e.g. about 0.5 to about 5% w/w, about 5 to about 10% w/w, about 1 to about 4% w/w, about 1 to about 8% w/w, about 2 to about 7% w/w, about 3 to about 6% w/w, about 4 to about 8% w/w, or about 6 to about 10% w/w. In some embodiments, the amphiphilic compound of the formula (I) is present in the pharmaceutical formulation in an amount of 0.1% w/w, 0.2% w/w, 0.3% w/w, 0.4% w/w, 0.5% w/w, 0.6% w/w, 0.7% w/w, 0.8% w/w, 0.9% w/w, 1% w/w, 2% w/w, 3% w/w, 4% w/w, 5% w/w, 6% w/w, 7% w/w, 8% w/w, 9% w/w, or 10% w/w.

In some embodiments, the amphiphilic compound of the formula (I) is a bile acid or bile salt, or a derivative or metabolite thereof. The bile acid or bile salt may be a natural bile acid or bile salt, a synthetic bile acid or bile salt, or a semi-synthetic (modified) bile acid or bile salt.

Bile acids are derivatives of cholesterol which are synthesised in the liver. Bile acids are critical for digestion and absorption of fats and fat-soluble vitamins in the small intestine. Cholesterol, ingested as part of the diet or derived from hepatic synthesis, is converted into the bile acids, cholic and chenodeoxycholic acids, which are then conjugated to an amino acid (glycine or taurine) to yield the conjugated form.

Set out below are two schematic pathways (Scheme 1 and Scheme 2) detailing naturally-produced bile acids in mammals.

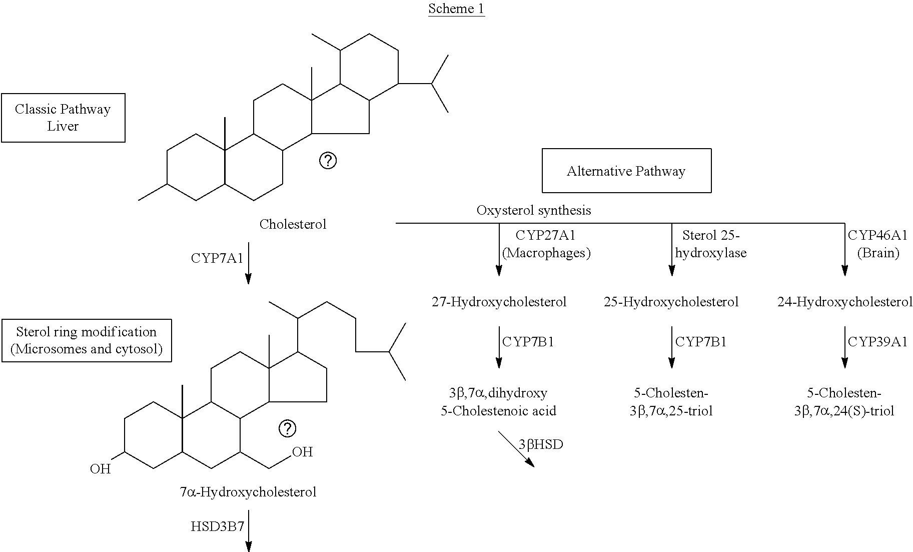

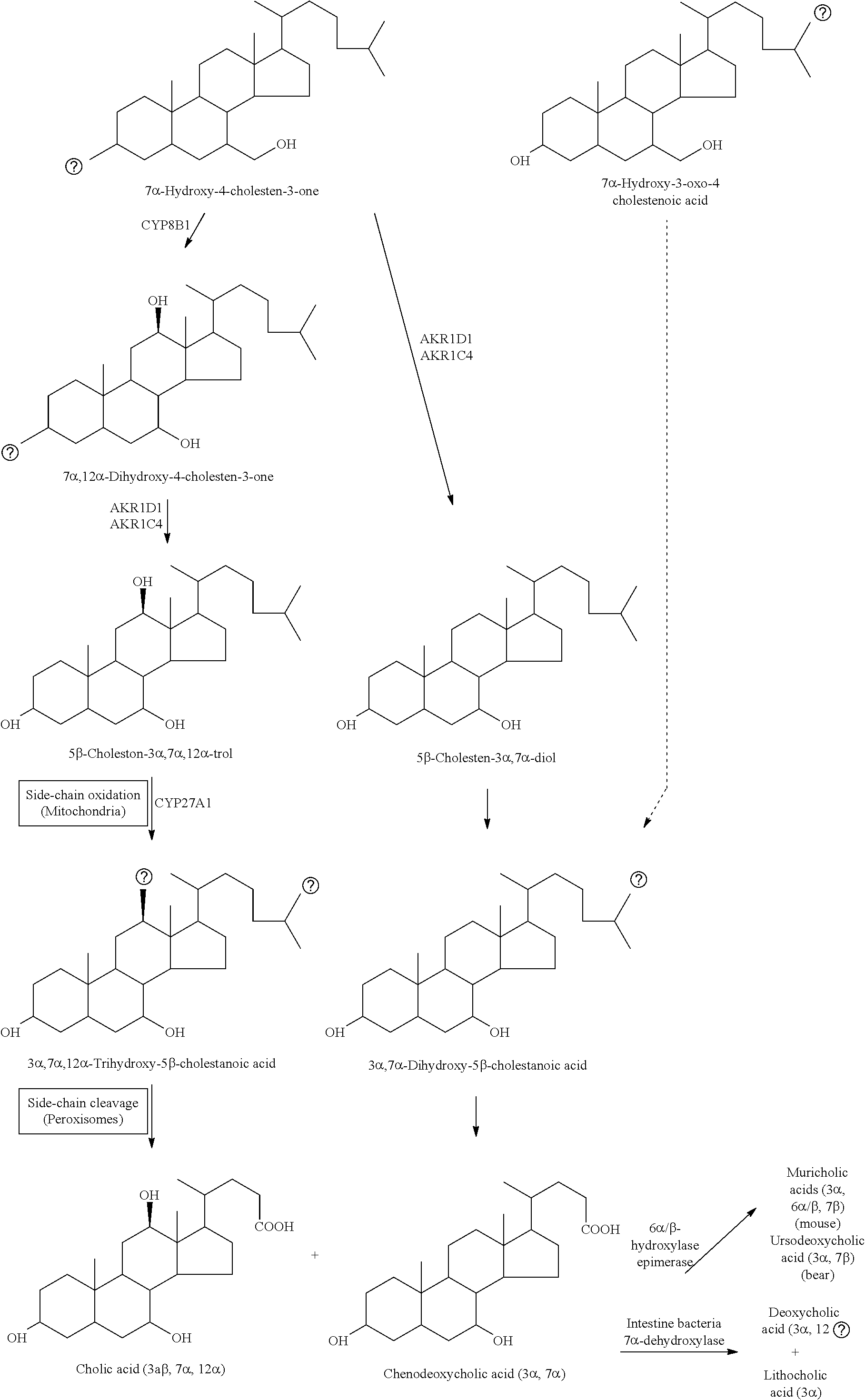

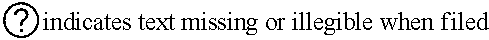

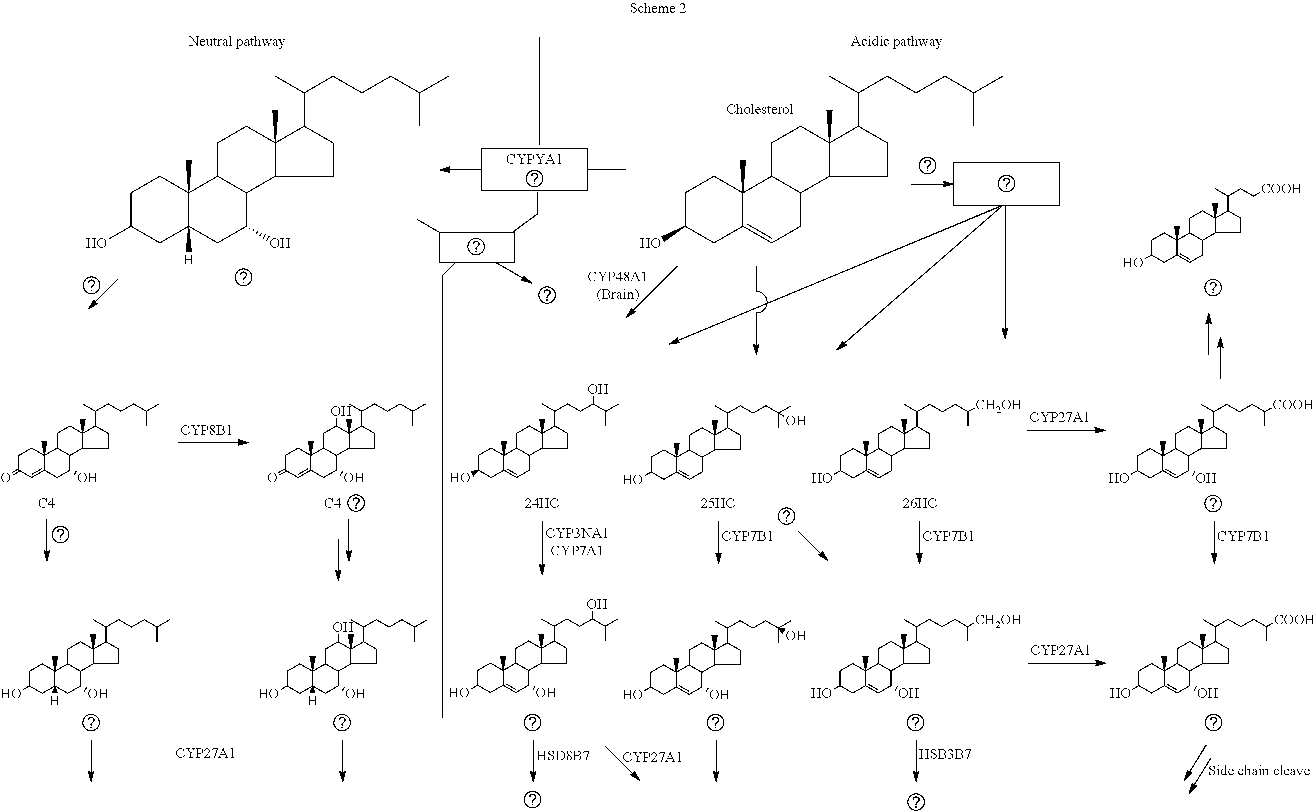

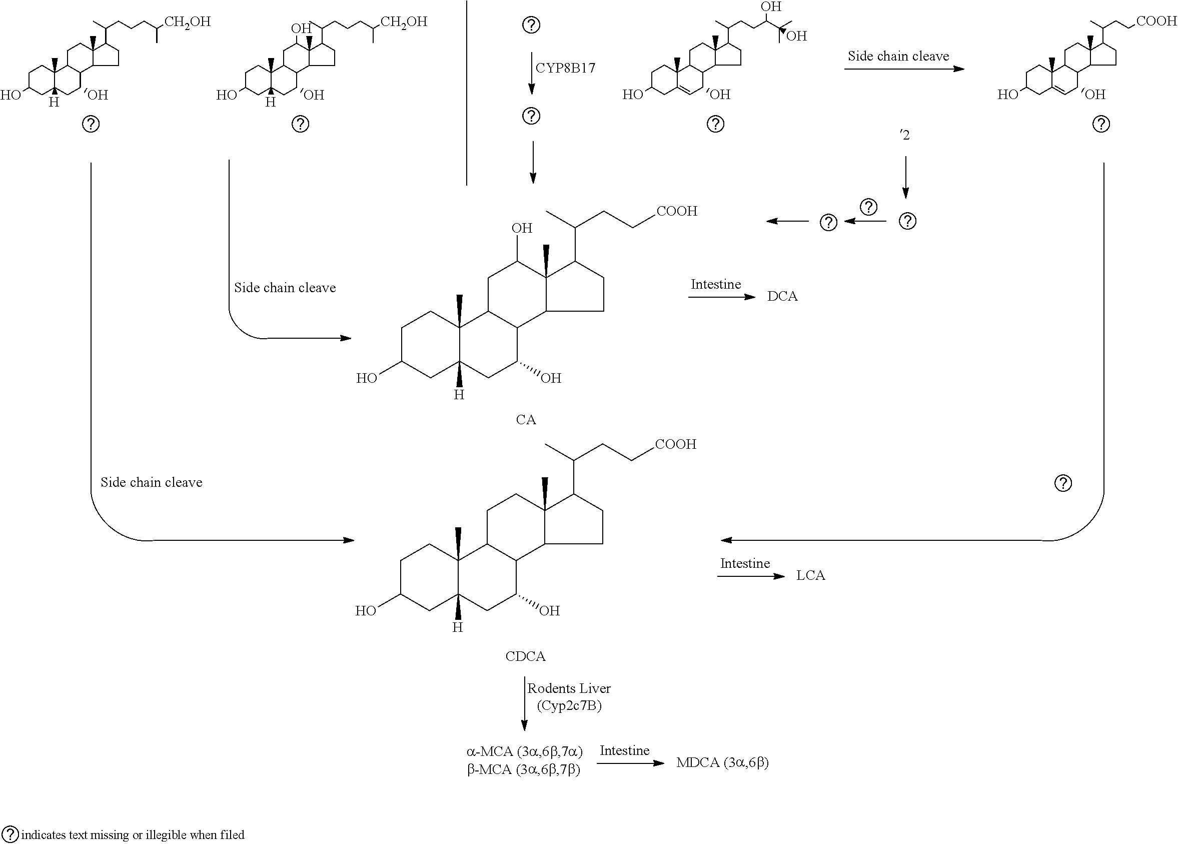

Bile acids are facial amphiphiles, i.e. they contain both hydrophobic and hydrophilic faces. The cholesterol-derived portion of a bile acid has one face that is hydrophobic (with methyl groups) and one that is hydrophilic (with the hydroxyl groups); the amino acid conjugate is polar and hydrophilic.

Being facial amphiphiles has enabled bile acids to be employed in drug delivery systems for selective drug-targeting to the liver or to enhance drug bioavailability by improving intestinal absorption and metabolic stability. Bile acid-based drug delivery systems, in the form of mixed micelles, bilosomes and drug conjugates, are versatile nanocarriers.

Any bile acid or bile salt, or a derivative or metabolite thereof, may be used to enhance the bioavailability of the active ingredient, and may be in the form of bile acid-based nanoparticles or bile acid-based microparticles. For example, the bile acid or bile salt may be a primary bile acid, or a secondary bile acid or any bile acid with an amino acid moiety attached. Various bile acids and bile salts are commercially available. For example, the bile acid or bile salt may be selected from deoxycholic acid (DCA), ursodeoxycholic acid (UDCA; also known as ursodiol), cholic acid, taurocholic acid, glycocholic acid, glycodeoxycholic acid, glycochenodeoxycholic acid, taurodeoxycholic acid, taurochenodeoxycholic acid, lithocholic acid, glycolithocholic acid, chenodeoxycholic acid, taurolithocholic acid, tauroursodeoxycholic acid, obeticholic acid, all muricholic acids (α-muricholic acid, β-muricholic acid, γ-muricholic acid and ω-muricholic acid), glycomuricholic acid, tauromuricholic acid, or a salt, derivative or metabolite thereof. In some embodiments, the bile acid is deoxycholic acid. In some embodiments, the bile acid is ursodeoxycholic acid. In some embodiments, both deoxycholic acid and ursodeoxycholic acid are used. In some embodiments, the bile acid is lithocholic acid. In some embodiments, the bile acid is taurolithocholic acid. In some embodiments, the bile acid is tauroursodeoxycholic acid. In some embodiments, the bile acid is glycocholic acid.

In some embodiments, the bile acid or bile salt, or a derivative or metabolite thereof, is present in the pharmaceutical formulation in an amount of from about 0.1% w/w to about 10% w/w, e.g. about 0.5 to about 5% w/w, about 5 to about 10% w/w, about 1 to about 4% w/w, about 1 to about 8% w/w, about 2 to about 7% w/w, about 3 to about 6% w/w, about 4 to about 8% w/w, or about 6 to about 10% w/w. In some embodiments, the bile acid or bile salt is present in the pharmaceutical formulation in an amount of 0.1% w/w, 0.2% w/w, 0.3% w/w, 0.4% w/w, 0.5% w/w, 0.6% w/w, 0.7% w/w, 0.8% w/w, 0.9% w/w, 1% w/w, 2% w/w, 3% w/w, 4% w/w, 5% w/w, 6% w/w, 7% w/w, 8% w/w, 9% w/w, or 10% w/w.

In some embodiments, the amphiphilic compound of the formula (I) is a bile acid selected from deoxycholic acid, cholic acid, taurocholic acid, glycocholic acid, glycodeoxycholic acid, taurodeoxycholic acid, ursodeoxycholic acid, taurochenodeoxycholic acid, lithocholic acid, glycolithocholic acid, chenodeoxycholic acid, taurolithocholic acid, tauroursodeoxycholic acid, obeticholic acid, any muricholic acid (e.g. α-muricholic acid, β-muricholic acid, γ-muricholic acid, ω-muricholic acid), glycomuricholic acid, tauromuricholic acid, or glycochenodeoxycholic acid, or a salt thereof. In some embodiments, the amphiphilic compound of the formula (I) is deoxycholic acid. In some embodiments, the amphiphilic compound of the formula (I) is ursodeoxycholic acid. In some embodiments, the amphiphilic compound of the formula (I) is lithocholic acid.

In some embodiments, two of more amphiphilic compounds of the formula (I) may be used. In some embodiments, both deoxycholic acid and ursodeoxycholic acid are used.

Advantageously, the amphiphilic compound of the formula (I), (e.g. a bile acid or bile salt, or a derivative or metabolite thereof) in the formulation enhances the bioavailability of the active ingredient by acting as a permeability enhancer. Typically, the amphiphilic compounds of the formula (I) are facial amphiphiles that can act as permeability enhancers.

Advantageously, the amphiphilic compound of the formula (I) enhances permeation through the middle ear (tympanic membrane) into the round window and inner ear (cochlea, Organ of Corti, Scala vestibule, Scala tympani and Scala media). Drug permeation in sufficient amounts deep into the cochlea is one of the major challenges of current commercial gels. Additionally, the amphiphilic compound of the formula (I) may function as a stabilising agent, or as a component that may provide slow or controlled release of the active ingredient.

In some embodiments, the pharmaceutical formulations comprising such permeability enhancers will facilitate the delivery of the composition across biological barriers that separate the middle and inner ear, e.g., the round window, thereby efficiently delivering a therapeutically effective amount of the pharmaceutical formulation to the inner ear. Efficient delivery to the cochlea, Organ of Corti, and/or vestibular organs is desired because these tissues host the support cells that promote sensory hair cell regeneration when treated or contacted with formulations described herein.

In some embodiments, the agent that enhances the bioavailability of the active ingredient comprises a further substance that is a permeability enhancer.

In some embodiments, the further permeability enhancer is selected from a polyol such as polyethylene glycol (PEG), glycerol (glycerin), maltitol, sorbitol, etc.; diethylene glycol monoethyl ether, azone, benzalkonium chloride (ADBAC), cetylperidium chloride, cetylmethylammonium bromide, dextran sulfate, lauric acid, menthol, methoxy salicylate, oleic acid, phosphatidylcholine, polyoxyethylene, polysorbate 80, sodium glycholate, sodium lauryl sulfate, sodium salicylate, sodium taurocholate, sodium taurodeoxycholate, sulfoxides, sodium deoxycholate, sodium glycodeoxycholate, sodium taurocholate and surfactants such as sodium lauryl sulfate, laureth-9, cetylpyridinium chloride and polyoxyethylene monoalkyl ethers, benzoic acids, such as sodium salicylate and methoxy salicylate, fatty acids, such as lauric acid, oleic acid, undecanoic acid and methyl oleate, fatty alcohols, such as octanol and nonanol, laurocapram, cyclodextrins, thymol, limonene, urea, chitosan and other natural and synthetic polymers.

In some embodiments, the further permeability enhancer is present in the pharmaceutical formulation in an amount of from about 0.1% w/w to about 10% w/w, e.g. about 0.5 to about 5% w/w, about 5 to about 10% w/w, about 1 to about 4% w/w, about 1 to about 8% w/w, about 2 to about 7% w/w, about 3 to about 6% w/w, about 4 to about 8% w/w, or about 6 to about 10% w/w. In some embodiments, the permeability enhancer is present in the pharmaceutical formulation in an amount of 0.1% w/w, 0.2% w/w, 0.3% w/w, 0.4% w/w, 0.5% w/w, 0.6% w/w, 0.7% w/w, 0.8% w/w, 0.9% w/w, 1% w/w, 2% w/w, 3% w/w, 4% w/w, 5% w/w, 6% w/w, 7% w/w, 8% w/w, 9% w/w, or 10% w/w.

In some embodiments, the further permeability enhancer is a cyclic oligosaccharide, which consists of a macrocyclic ring of glucose subunits joined by α-1,4 glycosidic bonds.

In some embodiments, the cyclic oligosaccharide is a cyclodextrin. Cyclodextrins are a family of cyclic oligosaccharides, consisting of a macrocyclic ring composed of five or more α-D-glucopyranoside units joined by α-1,4 glycosidic bonds. Typical cyclodextrins contain a number of glucose monomers ranging from six to eight units in a ring: α-cyclodextrin (6 glucose subunits), β-cyclodextrin (7 glucose subunits), γ-cyclodextrin (8 glucose subunits). Any cyclodextrin or a derivative thereof that enhances permeability may be used.

In some embodiments, the cyclodextrin may be an α-cyclodextrin, a β-cyclodextrin, or a γ-cyclodextrin, or a larger polymerised cyclodextrin. For example, the cyclodextrin may be (2-hydroxypropyl)-β-cyclodextrin.

Cyclodextrins have a hydrophobic interior and hydrophilic exterior, and form complexes with hydrophobic compounds such as hydrophobic active ingredients (e.g. probucol).

Advantageously, cyclodextrins may confer solubility and stability to the active ingredient, enabling the complexes of cyclodextrins with a hydrophobic active ingredient to be able to penetrate body tissues and release the active ingredient.

In some embodiments, the cyclodextrin is present in the pharmaceutical formulation in an amount of from about 1% w/w to about 50% w/w. For example, the cyclodextrin may be present in the pharmaceutical formulation in an amount of from about 1% w/w to about 25% w/w, e.g. from about 2% w/w to about 20% w/w, from about 3% w/w to about 15% w/w, or from about 5% w/w to about 10% w/w.

The salts of the amphiphilic compounds of the formula (I) are pharmaceutically acceptable. When amphiphilic compounds of the formula (I) contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydroiodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, e.g., Berge et al., Journal of Pharmaceutical Science 66:1-19 (1977)). Certain specific compounds of the present invention contain both basic and acidic functionalities that allow the compounds to be converted into either base or acid addition salts. Other pharmaceutically acceptable carriers known to those of skill in the art are suitable for the present invention.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

The amphiphilic compounds of the formula (I) may be synthesised by methods known in the art of organic synthesis. Methods for optimizing reaction conditions, if necessary minimising competing by-products, are known in the art. Reaction optimisation and scale-up may advantageously utilize high-speed parallel synthesis equipment and computer-controlled microreactors (e.g. Design And Optimization in Organic Synthesis, 2^{nd} Edition, Carlson R, Ed, 2005; Elsevier Science Ltd.; Jähnisch, K et al, Angew. Chem. Int. Ed. Engl. 2004 43: 406; and references therein). Additional reaction schemes and protocols may be determined by the skilled artesian by use of commercially available structure-searchable database software, for instance, SciFinder® (CAS division of the American Chemical Society) and CrossFire Beilstein® (Elsevier MDL), or by appropriate keyword searching using an internet search engine such as Google® or keyword databases such as the US Patent and Trademark Office text database. The invention includes the intermediate compounds used in making the compounds of the formulae herein as well as methods of making such compounds and intermediates, including without limitation those as specifically described in the examples herein.

The amphiphilic compounds of the formula (I) may also contain linkages (e.g., carbon-carbon bonds) wherein bond rotation is restricted about that particular linkage, e.g. restriction resulting from the presence of a ring or double bond. Accordingly, all cis/trans and E/Z isomers are expressly included in the present invention. The compounds herein may also be represented in multiple tautomeric forms, in such instances, the invention expressly includes all tautomeric forms of the compounds described herein, even though only a single tautomeric form may be represented. All such isomeric forms of such compounds herein are expressly included in the present invention. All crystal forms and polymorphs of the compounds described herein are expressly included in the present invention. Also embodied are extracts and fractions comprising compounds of the invention. The term “isomers” is intended to include diastereoisomers, enantiomers, regioisomers, structural isomers, rotational isomers, tautomers, and the like. For compounds which contain one or more stereogenic centres, e.g., chiral compounds, the methods of the invention may be carried out with an enantiomerically enriched compound, a racemate, or a mixture of diastereomers.

Preferred enantiomerically enriched compounds have an enantiomeric excess of 50% or more, more preferably the compound has an enantiomeric excess of 60%, 70%, 80%, 90%, 95%, 98%, or 99% or more. In preferred embodiments, only one enantiomer or diastereomer of a chiral compound of the invention is administered to cells or a subject.

Although the amphiphilic compounds of the formula (I) may be synthesised by methods known in the art, in several instances, the amphiphilic compounds of the formula (I) (such as deoxycholic acid, cholic acid, taurocholic acid, glycocholic acid, glycodeoxycholic acid, taurodeoxycholic acid, ursodeoxycholic acid, taurochenodeoxycholic acid, lithocholic acid, glycolithocholic acid, chenodeoxycholic acid, taurolithocholic acid, tauroursodeoxycholic acid, obeticholic acid, any muricholic acid (e.g. α-muricholic acid, β-muricholic acid, γ-muricholic acid, ω-muricholic acid), glycomuricholic acid, tauromuricholic acid, or glycochenodeoxycholic acid, or a salt thereof) are commercially available in purified forms. In other instances, such as the various modified bile acids and analogues thereof discussed above, the compounds can be synthesised according to procedures set forth or readily derivable from the literature (e.g. from U.S. Pat. Nos. 5,641,767, 5,656,277, 5,610,151, 5,428,182, and 3,910,888, all of which are incorporated herein by reference).

(iii) Optional Pharmaceutically Acceptable Carriers

In some embodiments, the pharmaceutical formulation comprises a pharmaceutically acceptable carrier. The pharmaceutically acceptable carrier is selected depending on the mode of administration of the pharmaceutical formulation, e.g. transtympanic administration. In advantageous embodiments, the pharmaceutically acceptable carrier is not perceived as foreign by the ear.

In some embodiments, the pharmaceutically acceptable carrier is water. Typically, water is a component of the pharmaceutical formulation of the invention. In some embodiments, the water is deionised water or distilled water.

In some embodiments, the pharmaceutically acceptable carrier comprises a polymer, e.g., a hydrogel (a water-soluble gel), that provides local and sustained release of the active ingredient (e.g. probucol). Such polymers and hydrogels are known in the art and are suitable for transtympanic administration. In some embodiments, the viscosity of the gel is 1-100 mPa S.

Examples of polymers and hydrogels suitable for transtympanic administration include: thermo-reversible triblock copolymer poloxamer 407 (see, e.g., Wang et al., Audiol Neurootol. 2009; 14(6):393-401. Epub 2009 Nov. 16; and Wang et al., Laryngoscope. 2011 February; 121(2):385-91); poloxamer-based hydrogels; Pluronic F-127 (see, e.g., Escobar-Chavez et al., J Pharm Pharm Sci. 2006; 9(3):339-5); Pluronic F68, F88, or F108; polyoxyethylene-polyoxypropylene triblock copolymer (e.g., a polymer composed of polyoxypropylene and polyoxyethylene, of general formula E106 P70 E106; see GB2459910, US20 110319377 and US20100273864); MPEGPCL diblock copolymers (Hyun et al., Biomacromolecules. 2007 April; 8(4):1093-100. Epub 2007 Feb. 28); hyaluronic acid hydrogels (Borden et al., Audiol Neurootol. 2011; 16(1):1-11); gelfoam cubes (see, e.g., Havenith et al., Hearing Research, February 2011; 272(1-2):168-177); and gelatin hydrogels (see, e.g., Inaoka et al., Acta Otolaryngol. 2009 April; 129(4):453-7).

Other biodegradable, biocompatible polymers may be used, such as ethylene vinyl acetate, polyanhydrides, polyglycolic acid, collagen, polyorthoesters, and polylactic acid. Tunable self-assembling hydrogels made from natural amino acids L and D may also be used, e.g., as described in Hauser et al e.g. Ac-LD6-COOH (L) e.g. Biotechnol Adv. 2012 May June; 30(3):593-603. Such formulations may be prepared using standard techniques, or obtained commercially, e.g., from Alza Corporation and Nova Pharmaceuticals, Inc.

In some embodiments, the pharmaceutically acceptable carrier is selected from one or more of glycerol, propylene glycol, polysorbate 80 (Tween 80), gels (e.g. water-soluble gels), hyaluronic acid, polyvinyl alcohol (PVA), poly-lactic-co-glycolic acid (PLGA), and PEG400. Thus, in some embodiments, the pharmaceutically acceptable carrier comprises any two or more of these carriers in combination. Such carriers are suitable for transtympanic administration.

In some embodiments, the pharmaceutically acceptable carrier is selected from glycerol, propylene glycol, and polysorbate 80 (Tween 80). In some embodiments, the pharmaceutically acceptable carrier is glycerol. In some embodiments, the pharmaceutically acceptable carrier is propylene glycol. In some embodiments, the pharmaceutically acceptable carrier is polysorbate 80 (Tween 80). In some embodiments, the pharmaceutically acceptable carrier is a water-soluble gel. In some embodiments, the pharmaceutical formulation comprises glycerol, propylene glycol, polysorbate 80 (Tween 80), and a water-soluble gel.

In some embodiments, the water-soluble gel is selected from a thermo-sensitive gel or an adhesive sol-gel transition hydrogel. Commercially available water-soluble gels suitable for use in the pharmaceutical formulation of the invention include, for example, Metron water-soluble ultrasonic gel, Pluronic F127 and hyaluronic acid (HA).

Accordingly, in some embodiments, the pharmaceutical formulation of the present invention may comprise an active ingredient (e.g. probucol or succinobucol) and any one or more of the following:
1: Polyethylene glycol (of any molecular weight) and any derivative thereof;
2: Polyvinylpyrrolidone (of any molecular weight) and any derivative thereof;
3: Polytetrafluoroethylene (PTFE; Teflon) (of any molecular weight) and any derivative thereof;
4: Poly-L-Ornithine (of any molecular weight) and any derivative thereof;
5: Polystyrene sulphonate (of any molecular weight) and any derivative thereof;
6: Acacia (of any molecular weight);
7: Starch (of any molecular weight);
8: Chitosan (of any molecular weight);
9: Poly-L-lysine (of any molecular weight) and any derivative thereof;
10: Poly-allyl-amine (of any molecular weight) and any derivative thereof;
11: Gelatin (of any molecular weight) and any derivative thereof;
12: Pectin (of any molecular weight) and any derivative thereof;
13: Poly (vinyl alcohol) (of any molecular weight) and any derivative thereof;
14: Poloxamer 407;
15: Polysorbate of any polyoxyethylene number and configuration;
16: Pluronic F127;
17: Hyaluronic acids (of any molecular weight) and any derivative thereof;
18: Alginates of any G and M unit varieties/combinations and any derivative thereof;
19: Bile acids: including salts with any cation as well as conjugation with any amino acid (natural, synthetic and derivatives of);
20: Spiroquinone: (chemical name: 2,4,9,11-tetrakis(1,1-dimethylethyl)-14,14-dimethyl-13,15-dithiadispiro [5.0.5.3] pentadeca-1,4,8,11-tetraene-3,10-dione); 21: All cyclodextrins and derivates thereof: alpha-, beta-, and gamma-cyclodextrin and any of their derivatives: any cyclodextrin including any member of the family of cyclic oligosaccharides, consisting of a macrocyclic ring of glucose subunits joined by α-1,4 glycosidic bonds. These include all cyclodextrins composed of 5 or more α-D-glucopyranoside units linked 1->4, as in amylose with any number of glucose monomers (as an example: α (alpha)-cyclodextrin: 6 glucose subunits, β (beta)-cyclodextrin: 7 glucose subunits and γ (gamma)-cyclodextrin: 8 glucose subunits) and also all their derivatives including any modification to their hydroxyl group (including (2-Hydroxypropyl)-β-cyclodextrin and all derivatives thereof);
22: Propylene glycol: propane-1,2-diol; or
23: Triethanolamine.

In some embodiments, the pharmaceutical formulation comprising the active ingredient, an agent that enhances the bioavailability of the active ingredient, and optionally a pharmaceutically acceptable carrier, is in the form of a solution, a mixture, a suspension, microparticles, nanoparticles, or a gel.

When the pharmaceutical formulation is in form of a solution, the concentration of the active ingredient may, for example, be from 0.1 to 1000 mg/mL, e.g. from 0.1 to 2 mg/mL, from 0.2 to 5 mg/mL, from 1 to 5 mg/mL, from 2 to 8 mg/mL, from 5 to 10 mg/mL, from 10 to 20 mg/mL, from 20 to 30 mg/mL, from 30 to 40 mg/mL, from 40 to 50 mg/mL, from 50 to 60 mg/mL, from 60 to 70 mg/mL, from 70 to 80 mg/mL, from 80 to 90 mg/mL, from 90 to 100 mg/mL, from 10 to 100 mg/mL, from 100 to 200 mg/mL, from 200 to 300 mg/mL, from 300 to 400 mg/mL, from 500 to 1000 mg/mL, from 400 to 500 mg/mL, from 500 to 600 mg/mL, from 600 to 700 mg/mL, from 700 to 800 mg/mL, from 800 to 900 mg/mL, or from 900 to 1000 mg/mL of the active ingredient.

When the pharmaceutical formulation is in form of a suspension, the concentration of the active ingredient may, for example, be from 0.1 to 1000 mg/mL, e.g. from 0.1 to 2 mg/mL, from 0.2 to 5 mg/mL, from 1 to 5 mg/mL, from 2 to 8 mg/mL, from 5 to 10 mg/mL, from 10 to 20 mg/mL, from 20 to 30 mg/mL, from 30 to 40 mg/mL, from 40 to 50 mg/mL, from 50 to 60 mg/mL, from 60 to 70 mg/mL, from 70 to 80 mg/mL, from 80 to 90 mg/mL, from 90 to 100 mg/mL, from 10 to 100 mg/mL, from 100 to 200 mg/mL, from 200 to 300 mg/mL, from 300 to 400 mg/mL, from 500 to 1000 mg/mL, from 400 to 500 mg/mL, from 500 to 600 mg/mL, from 600 to 700 mg/mL, from 700 to 800 mg/mL, from 800 to 900 mg/mL, or from 900 to 1000 mg/mL of the active ingredient.

When the pharmaceutical formulation is in form of a microparticles, the concentration of the active ingredient may, for example, be from 0.1 to 1000 mg/mL, e.g. from 0.1 to 2 mg/mL, from 0.2 to 5 mg/mL, from 1 to 5 mg/mL, from 2 to 8 mg/mL, from 5 to 10 mg/mL, from 10 to 20 mg/mL, from 20 to 30 mg/mL, from 30 to 40 mg/mL, from 40 to 50 mg/mL, from 50 to 60 mg/mL, from 60 to 70 mg/mL, from 70 to 80 mg/mL, from 80 to 90 mg/mL, from 90 to 100 mg/mL, from 10 to 100 mg/mL, from 100 to 200 mg/mL, from 200 to 300 mg/mL, from 300 to 400 mg/mL, from 500 to 1000 mg/mL, from 400 to 500 mg/mL, from 500 to 600 mg/mL, from 600 to 700 mg/mL, from 700 to 800 mg/mL, from 800 to 900 mg/mL, or from 900 to 1000 mg/mL of the active ingredient.

When the pharmaceutical formulation is in form of a nanoparticles, the concentration of the active ingredient may, for example, be from 0.1 to 1000 mg/mL, e.g. from 0.1 to 2 mg/mL, from 0.2 to 5 mg/mL, from 1 to 5 mg/mL, from 2 to 8 mg/mL, from 5 to 10 mg/mL, from 10 to 20 mg/mL, from 20 to 30 mg/mL, from 30 to 40 mg/mL, from 40 to 50 mg/mL, from 50 to 60 mg/mL, from 60 to 70 mg/mL, from 70 to 80 mg/mL, from 80 to 90 mg/mL, from 90 to 100 mg/mL, from 10 to 100 mg/mL, from 100 to 200 mg/mL, from 200 to 300 mg/mL, from 300 to 400 mg/mL, from 500 to 1000 mg/mL, from 400 to 500 mg/mL, from 500 to 600 mg/mL, from 600 to 700 mg/mL, from 700 to 800 mg/mL, from 800 to 900 mg/mL, or from 900 to 1000 mg/mL of the active ingredient.

When the pharmaceutical formulation is in form of a gel, the concentration of the active ingredient may, for example, be from 0.1 to 1000 mg/mL, e.g. from 0.1 to 2 mg/mL, from 0.2 to 5 mg/mL, from 1 to 5 mg/mL, from 2 to 8 mg/mL, from 5 to 10 mg/mL, from 10 to 20 mg/mL, from 20 to 30 mg/mL, from 30 to 40 mg/mL, from 40 to 50 mg/mL, from 50 to 60 mg/mL, from 60 to 70 mg/mL, from 70 to 80 mg/mL, from 80 to 90 mg/mL, from 90 to 100 mg/mL, from 10 to 100 mg/mL, from 100 to 200 mg/mL, from 200 to 300 mg/mL, from 300 to 400 mg/mL, from 500 to 1000 mg/mL, from 400 to 500 mg/mL, from 500 to 600 mg/mL, from 600 to 700 mg/mL, from 700 to 800 mg/mL, from 800 to 900 mg/mL, or from 900 to 1000 mg/mL of the active ingredient.

The pharmaceutical formulations are formulated to be compatible with the intended route of administration.

In one embodiment, the pharmaceutical formulation comprises a cyclodextrin (e.g. (2-hydroxypropyl)-β-cyclodextrin), a bile acid or bile salt (e.g. deoxycholic acid, cholic acid, taurocholic acid, glycocholic acid, glycodeoxycholic acid, taurodeoxycholic acid, ursodeoxycholic acid, taurochenodeoxycholic acid, lithocholic acid, glycolithocholic acid, chenodeoxycholic acid, taurolithocholic acid, tauroursodeoxycholic acid, obeticholic acid, any muricholic acid (e.g. α-muricholic acid, β-muricholic acid, γ-muricholic acid, ω-muricholic acid), glycomuricholic acid, tauromuricholic acid, or glycochenodeoxycholic acid, or a salt thereof), and the active ingredient, in a form suitable for cochlea (inner ear) delivery.

In one embodiment, the pharmaceutical formulation comprises a cyclodextrin (e.g. (2-hydroxypropyl)-@-cyclodextrin), a bile acid or bile salt (e.g. deoxycholic acid, cholic acid, taurocholic acid, glycocholic acid, glycodeoxycholic acid, taurodeoxycholic acid, ursodeoxycholic acid, taurochenodeoxycholic acid, lithocholic acid, glycolithocholic acid, chenodeoxycholic acid, taurolithocholic acid, tauroursodeoxycholic acid, obeticholic acid, any muricholic acid (e.g. α-muricholic acid, β-muricholic acid, γ-muricholic acid, ω-muricholic acid), glycomuricholic acid, tauromuricholic acid, or glycochenodeoxycholic acid, or a salt thereof), and the active ingredient, in a gel formulation suitable for cochlea (inner ear) delivery.

In one embodiment, the pharmaceutical formulation comprises (2-hydroxypropyl)-β-cyclodextrin, deoxycholic acid and/or ursodeoxycholic acid, and the active ingredient, in a gel formulation suitable for cochlea (inner ear) delivery.

In one embodiment, the pharmaceutical formulation comprises (2-hydroxypropyl)-β-cyclodextrin, deoxycholic acid, ursodeoxycholic acid, and the active ingredient, in a gel formulation suitable for cochlea (inner ear) delivery.

In one embodiment, the pharmaceutical formulation comprises a cyclodextrin, deoxycholic acid, and the active ingredient in the form of nanoparticles in an ultrasonic gel, for cochlea (inner ear) delivery.

In one embodiment, the pharmaceutical formulation comprises a cyclodextrin, deoxycholic acid, ursodeoxycholic acid, and the active ingredient in the form of nanoparticles in an ultrasonic gel, for cochlea (inner ear) delivery.

In one embodiment, the pharmaceutical formulation comprises a (2-hydroxypropyl)-β-cyclodextrin, deoxycholic acid, ursodeoxycholic acid, and the active ingredient in the form of nanoparticles in an ultrasonic gel, for cochlea (inner ear) delivery.

In some embodiments, a pharmaceutical formulation in the form of a gel may include the following components: probucol, one or more bile acids, one or more cyclodextrins, polysorbate 80 (Tween 80), glycerol, propylene glycol, a water-soluble gel, and water.

For example, the pharmaceutical formulation may include the following components:

| Component | Amount |
|---|---|
| Water | about 200-500 mL |
| (2-Hydroxypropyl)-β-cyclodextrin | about 1-15 g |
| Polysorbate 80 (Tween 80) | about 1-10 mL |
| Glycerol | about 1-10 mL |
| Propylene glycol | about 1-20 mL |
| Metron Water Soluble Gel | about 1-20 g |
| Probucol | about 0.1-10 g |
| Deoxycholic acid (DCA) | about 0.1-10 g |
| Ursodeoxycholic acid (UDCA) | about 0.1-10 g |

Two specific formulation examples are provided below:

| Component | Amount | Amount |
|---|---|---|
| Water | about 350 mL | about 350 mL |
| (2-Hydroxypropyl)-β-cyclodextrin | about 7 g | about 7 g |
| Polysorbate 80 (Tween 80) | about 5 mL | about 5 mL |
| Glycerol | about 6 mL | about 5 mL |
| Propylene glycol | about 12 mL | about 11 mL |
| Metron Water Soluble Gel | about 6.5 g | about 6 g |
| Probucol | about 4 g | about 4 g |
| Deoxycholic acid (DCA) | about 3 g | about 2 g |
| Ursodeoxycholic acid (UDCA) | about 1 g | about 3 g |

A typical method of preparation is as follows. A mixture of (2-hydroxypropyl)-β-cyclodextrin in water is mixed with stirring at about 80° C. for about 1 hour. Probucol is then added and the resulting mixture is heated with stirring to about 80° C. for about 1 hour. Tween 80 is then added, and the resulting mixture is heated with stirring to about 80° C. for about 1 hour. Ursodeoxycholic acid (UDCA) is then added, and the resulting mixture is heated with stirring to about 80° C. for about 1 hour. Deoxycholic acid is then added, and the resulting mixture is heated with stirring to about 80° C. for about 1 hour. Metron Water Soluble Gel is then added, and the resulting mixture is heated with stirring to about 80° C. for about 1 hour. Glycerol and propylene glycol are then added, and the resulting mixture is heated with stirring to about 80° C. for about 1 hour. The resulting mixture is then stirred at about 80° C. for a further hour, and then water is added to make up the volume to the desired amount.

Processes for formulating suitable pharmaceutical formulations are known in the art, see, e.g., Remington: The Science and Practice of Pharmacy. 21st ed., 2005; and the books in the series Drugs and the Pharmaceutical Sciences: A Series of Textbooks and Monographs (Dekker, NY). For example, solutions or suspensions can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The pH may be adjusted with acids or bases, such as hydrochloric acid or sodium hydroxide. The parenteral preparation may be enclosed in ampoules, disposable syringes or multiple dose vials made of glass or plastic.

Pharmaceutical formulations suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the formulation must be sterile and should be fluid to the extent that easy syringability exists. It should be stable under the conditions of manufacture and storage and must be preserved against the contaminating action of microorganisms such as bacteria and fungi. The carrier may be a solvent or dispersion medium containing, for example, water, ethanol, polyol (for example, glycerol, propylene glycol, and liquid polyetheylene glycol, and the like), and suitable mixtures thereof. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the formulation. Prolonged absorption of the injectable formulations may be brought about by including in the formulation an agent that delays absorption, for example, aluminium monostearate and gelatin.

Sterile injectable solutions may be prepared by incorporating the active ingredient (e.g., probucol) in the required amount in an appropriate solvent with one or a combination of ingredients enumerated above, as required, followed by filtered sterilisation. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

In some embodiments, the pharmaceutical formulation is administered by inserting a foam-like formulation into the ear, and the eardrum gently peeled off to allow entry of the formulation, and then sealed back.

In some embodiments, the pharmaceutical formulation is a solid or semi-solid formulation. In some embodiments, the pharmaceutical formulation is in the form of a liquid, mixture, suspension, emulsion, gel, foam or solution.

In some embodiments, the pharmaceutical formulation is administered by one of the modes of delivery listed below:
incorporated into or used in conjunction with tympanic membrane graft material as part of tympanoplasty or myringoplasty surgery;
incorporated into middle ear prostheses as part of ossicular chain reconstructive surgery;
used with established middle ear packing material such as Gelfoam or Gelfilm;
used as part of irrigation solution during middle ear or inner ear surgical procedures;
incorporated with suture material;
incorporated into ventilation tubes (grommets);
incorporated into bone condition implants such as the BAHA, Bonebridge, Ossia or Ponto device; or
incorporated into microsurgical drills.

In some embodiments, the pharmaceutical formulation may comprise one or more components of the formulation described above in Section 2.1, or be administered by a mode of delivery to the outer, middle or inner ear described above in Section 2.1.

The pharmaceutical formulations may be included in a container, pack, or dispenser together with instructions for administration.

### 3. Hearing Impairment

Hearing and balance impairment often involves the loss of sensory or neural cells, such as cochlear and vestibular hair cells and afferent neurons, but can include damaged accessory structures such as stria vascularis or cells in the spiral ligament, supporting cells (cells supporting hair cells), microvasculature endothelium, or the endolymphatic duct and sac. This cellular loss or damage can either be due to the primary insult (i.e. direct cell loss caused by noise trauma or virus), though in many cases involves progressive cellular damage due to molecular mechanisms bringing about cellular apoptosis, driven by high levels of free-radicals, oxidative stress, and pro-inflammatory cytokines. Hearing loss is a diminished ability to perceive sounds relative to normative levels. This may be caused either by a conductive hearing loss, sensorineural hearing loss, or a combination of both. Sensorineural hearing loss is caused by dysfunction of the sensory and/or neural cells of the cochlea, and/or dysfunction of the specialized epithelia in the inner ear such as stria vacularis and cochlear supporting cells.

In some embodiments, hearing loss to be prevented or treated by the methods of the present invention is caused by a noise trauma, by a medical intervention, by ischemic injury, by age or is chemically induced. The hearing loss may thus be a consequence of a medical intervention such as e.g. cochlear implantation. The chemical induction is usually caused by a chemical agent e.g. by an antibiotic or a chemotherapeutic agent. In some preferred embodiments hearing loss is sudden hearing loss. Hearing loss caused by age comprises, for example, presbycusis. In some embodiments, hearing loss caused by a noise trauma, cochlear implantation, or which is chemically induced, e.g. by an antibiotic, is prevented or treated by the methods of the present invention. In some embodiments, hearing loss caused by a noise trauma or which is chemically induced, e.g. by an antibiotic, is prevented or treated by the methods of the present invention. In some embodiments, hearing loss is of sensorineural origin caused by a damage leading to malnutrition of the cells in early brain development. In this case, early treatment with probucol or other suitable active ingredient may be disease-modifying, thereby preventing further damage.

In some embodiments, the hearing disorder is selected from sudden deafness, blast-induced hearing loss, ototoxicity, ARHL (age-related hearing loss, i.e. presbycusis) and noise damage.

### Hearing Loss, Hair Cell Degeneration or Hair Cell Death Caused by a Noise Trauma or by Medical Intervention

Exposure to loud noise causes noise-induced hearing loss (NIHL) by damaging the organ of Corti. Damage by NIHL depends upon both the level of the noise and the duration of the exposure. Hearing loss may be temporary (temporary threshold shift, TTS) if a repair mechanism is able to restore the Organ of the Corti. However, it becomes permanent (permanent threshold shift, PTS) when hair cells or neurons die. Structural modifications correlated to noise trauma are of two types: (1) mild damage of synapses and or hair cell stereocilia which may be repaired by cellular repair mechanisms and accounts for TTS and recovery and (2) severe damage inducing hair cell and neuronal apoptosis which cannot be repaired by cellular repair mechanisms and accounts for PTS.

A noise trauma as referred herein is a noise trauma which is sufficient to cause damage to the organ of Corti, in particular a noise trauma causing temporary or permanent hearing loss. A noise trauma may be caused by exposure to a sound pressure level of e.g., at least 70 dB (SPL), at least 90 dB (SPL), at least 100 dB (SPL), at least 120 dB (SPL) or at least 130 dB (SPL).

Hearing loss can also be caused by a medical intervention usually by a medical intervention in the ear e.g. by cochlea surgery such as cochlear implantation.

In some embodiments the pharmaceutical formulation of the invention is administered before the subject is exposed to a noise trauma or medical intervention. In some embodiments, the pharmaceutical formulation of the invention is administered after the subject is exposed to a noise trauma or medical intervention. In a particular embodiment the pharmaceutical formulation of the invention is administered prior to cochlear surgery i.e. before the subject undergoes cochlear surgery.

### Hearing Loss, Hair Cell Degeneration or Hair Cell Death Caused by Age

Hearing loss caused by age also referred in the literature as “age-related hearing loss” is the cumulative effect of aging on hearing. It is normally a progressive bilateral symmetrical age-related sensorineural hearing loss. The hearing loss is most marked at higher frequencies.

There are four pathological types of hearing loss caused by age:
1) sensory: characterised by degeneration of Organs of Corti.
2) neural: characterised by degeneration of cells of spiral ganglion.
3) strial/metabolic: characterised by atrophy of the stria vascularis in all turns of cochlea.
4) cochlear conductive: due to stiffening of the basilar membrane thus affecting its movement.

Hearing loss caused by age to be prevented or treated by the methods of the present invention is usually related to the first pathological type i.e. hearing loss characterised by degeneration of the Organ of Corti. Thus, in some embodiments the pharmaceutical formulation of the invention is administered to the subject prior to degeneration of the Organ of Corti, e.g. prior to damage or apoptosis of hair cells and/or prior to hair cell degeneration or hair cell death.

### Chemically Induced Hearing Loss, Hair Cell Degeneration or Hair Cell Death

Hearing loss, hair cell degeneration or hair cell death may be induced chemically, i.e. by a chemical agent. Examples of types of chemical agents that can induce hearing loss, hair cell degeneration or hair cell death include antibiotics, drugs, chemotherapeutic agents, heavy metals, and organic agents. Antibiotics which may cause hearing loss include, for example, cephalosporins such as cephalexin (Keflex), cefaclor (Ceclor), and cefixime (Suprax); aminoglycosides such as gentamycin, tobramycin and streptomycin; macrolides, such as erythromycin, azithromycin (Zithromax) and clarithromycin; sulfonamides such as trimethoprim-sulfamethoxazole or tetracylines such as tetracycline, or doxycycline. In particular, hearing loss, hair cell degeneration or hair cell death is effectively prevented or treated by the methods of the present invention in a subject exposed to gentamycin.

Chemotherapeutic agents, e.g. anti-cancer agents which may cause hearing loss, hair cell degeneration or hair cell death include for example platinum-containing agents e.g. cisplatin, and carboplatin, preferably cisplatin. Drugs which may cause hearing loss, hair cell degeneration or hair cell death include for example furosemide, quinine, aspirin and other salicylates. Heavy metals which may cause hearing loss include for example mercury, lead.

Organic agents which may cause hearing loss, hair cell degeneration or hair cell death include for example toluene, xylene, or styrene.

In some embodiments, the pharmaceutical formulation of the invention is administered to the subject before the subject is exposed to a chemical agent, thereby preventing the subject from chemically induced hearing loss, hair cell degeneration or hair cell death. In some embodiments, the pharmaceutical formulation of the invention is administered to the subject after the subject is exposed to a chemical agent thereby treating the subject having chemically induced hearing loss, hair cell degeneration or hair cell death.

In a preferred embodiment, when hearing loss is caused by a noise trauma or is chemically induced, the pharmaceutical formulation of the invention is administered to the subject prior to exposure of the subject to the noise trauma or to the chemical wherein there is prevention of at least 50%, at least 60%, at least 70%, at least 80%, at least 90%, at least 95%, at least 99%, or 100% of the cell damage of the hair cells caused by the noise trauma or the chemical agent.

The following examples are included to increase the understanding of the invention, without having any limiting effect of the invention.

### EXAMPLES

The present invention is further described below by reference to the following non-limiting Examples.

### Materials and Methods

All chemical reagents were purchased from commercial sources (Sigma Aldrich, Scharlab SL and Merck & Co, Inc.) and used without further purification.

### Formulation Example 1

Formulation Example 1 was prepared as follows, using the components listed in the table below.

| Component | Amount |
|---|---|
| Water | 350 mL |
| (2-Hydroxypropyl)-β-cyclodextrin | 7 g |
| Polysorbate 80 (Tween 80) | 5 mL |
| Glycerol | 6 mL |
| Propylene glycol | 12 mL |
| Metron Water Soluble Gel | 6.5 g |
| Probucol | 4 g |
| Deoxycholic acid (DCA) | 3 g |
| Ursodeoxycholic acid (UDCA) | 1 g |

### Method of Preparation:

A mixture of (2-hydroxypropyl)-@-cyclodextrin (7 g) was added to water (350 mL). The mixture was heated with stirring to 80° C. for 1 hour. Probucol (4 g) was added and the resulting mixture was heated with stirring to 80° C. for 1 hour. Tween 80 (5 mL) was then added and the resulting mixture was heated with stirring to 80° C. for 1 hour. Ursodeoxycholic acid (UDCA) (1 g) was then added and the resulting mixture was heated with stirring to 80° C. for 1 hour. Deoxycholic acid (3 g) was then added and the resulting mixture was heated with stirring to 80° C. for 1 hour. Metron Water Soluble Gel (6.5 g) was then added and the resulting mixture was heated with stirring to 80° C. for 1 hour. Glycerol (6 mL) and propylene glycol (12 mL) were then added and the resulting mixture was heated with stirring to 80° C. for 1 hour.

The resulting mixture was then stirred at 80° C. for a further hour, and then water was added to make the volume to 400 mL.

### Formulation Example 2

Formulation Example 2 was prepared as follows, using the components listed in the table below. This formulation resulted in the optimal balance of viscosity versus efficient drug loading and ideal fluid dynamics suitable for ear delivery.

| Component | Amount |
|---|---|
| Water | 350 mL |
| (2-Hydroxypropyl)-β-cyclodextrin | 7 g |
| Polysorbate 80 (Tween 80) | 5 mL |
| Glycerol | 5 mL |
| Propylene glycol | 11 mL |
| Metron Water Soluble Gel | 6 g |
| Probucol (“PB”) | 4 g |
| Deoxycholic acid (DCA) | 2 g |
| Ursodeoxycholic acid (UDCA) | 3 g |

### Method of Preparation:

A mixture of (2-hydroxypropyl)-@-cyclodextrin (7 g) was added to water (350 mL). The mixture was heated with stirring to 80° C. for 1 hour. Probucol (4 g) was added and the resulting mixture was heated with stirring to 80° C. for 1 hour. Tween 80 (5 mL) was then added and the resulting mixture was heated with stirring to 80° C. for 1 hour. Ursodeoxycholic acid (UDCA) (3 g) was then added and the resulting mixture was heated with stirring to 80° C. for 1 hour. Deoxycholic acid (2 g) was then added and the resulting mixture was heated with stirring to 80° C. for 1 hour. Metron Water Soluble Gel (6 g) was then added and the resulting mixture was heated with stirring to 80° C. for 1 hour. Glycerol (5 mL) and propylene glycol (11 mL) were then added and the resulting mixture was heated with stirring to 80° C. for 1 hour.

The resulting mixture was then stirred at 80° C. for a further hour, and then water was added to make the volume to 400 mL.

The resulting product was then used in the Experimental Examples without further purification.

### Experimental Examples

Experimental Examples 1, 2 and 3 described below used Formulation Example 1.

### Experimental Example 1: Assessing Probucol Formulation Permeation into the Cochlea Using Primary Mouse Cochlea

Experimental Example 1 describes ex vivo studies in healthy C₅₇ mice (standard type) and SAMP8 mice (age-accelerated) in which living cochlea of mice were removed, incubated in RPMI media and exposed to gel formulation for 12 hours.

Samples were left in a CO₂ incubator at 37° C. and gel permeation was ceased by removing cochlear samples, washing with phosphate buffered saline (PBS) and then sonicating with ice cold acetonitrile (HPLC grade, Thermo Fisher USA). The amount of probucol within the cochlea was measured, i.e. how much probucol had diffused or permeated into the deeper layers of the cochlea.

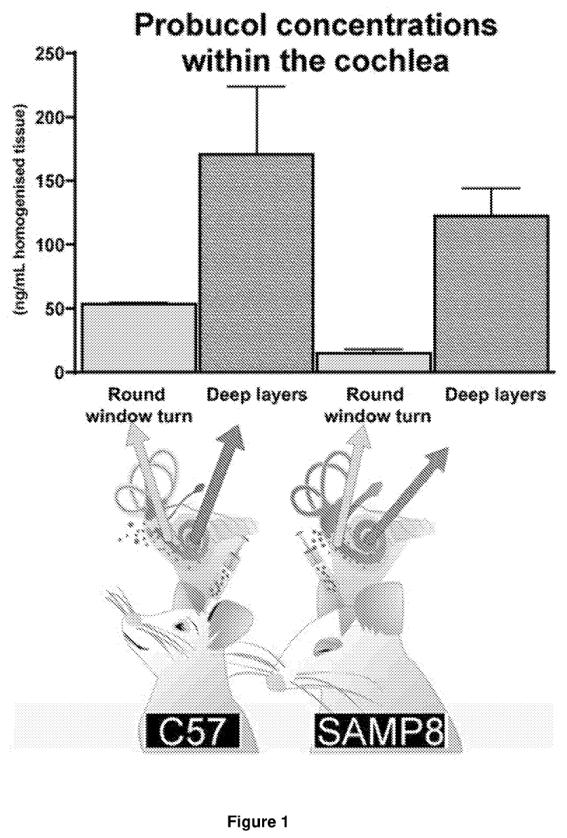

The results are shown in FIG. 1. “Round window turn” ‘in FIG. 1 indicates probucol levels within the inner ear immediately past (straight after) the round window of the cochlea where it was expected that most of the drug would be retained, while “Deep layers” indicates probucol levels deep in the cochlea. Thus, it was found that there was more probucol deep inside, than on the inner surface, which suggests that the formulation is unique in promoting probucol diffusion deeply into the inner ear.

### Experimental Example 2: Functional Testing of Formulation Example 2's Safety Profile on Auditory System (Animals Exposed to Large Dose of Probucol in Gel Formulation to Check Safety Profile)

Experimental Example 2 describes in vivo studies in healthy SD rats (standard type) in which hearing capacity was measured using Compound Action Potential (CAP) thresholds. Rats were anaesthetised (2% isoflurane in 98% oxygen; and artificially ventilated with temperature regulation), and the bullae was opened via a ventro-lateral approach to provide a view of the round window. A silver chloride electrode was placed on the bone of the 1^{st} cochlear turn for measuring CAP responses. CAP thresholds were measured using the rats' ears injected when the rats were anaesthetised and hearing measured in real time. CAPs evoked by 5 ms tone-burst (with a 1 ms rise/fall envelope) of 3, 6, 10, 14 and 18 kHz, at a range of sound levels were recorded and averaged, enabling estimation of the CAP thresholds from the response amplitude-level plots. That is, a linear regression line was fitted to the relationship between sound level and the peak to peak CAP amplitude. By extrapolating the regression line to a CAP peak to peak amplitude of zero, this provides the theoretical sound level for CAP threshold. A 20 μl volume of probucol (Formulation Example 2) was loaded onto the round window, whilst CAP thresholds were continuously monitored for. Baseline thresholds (pre-probucol) were taken for 45 minutes, and thresholds were monitored for 1.5 hours after the application of Probucol to the round window.

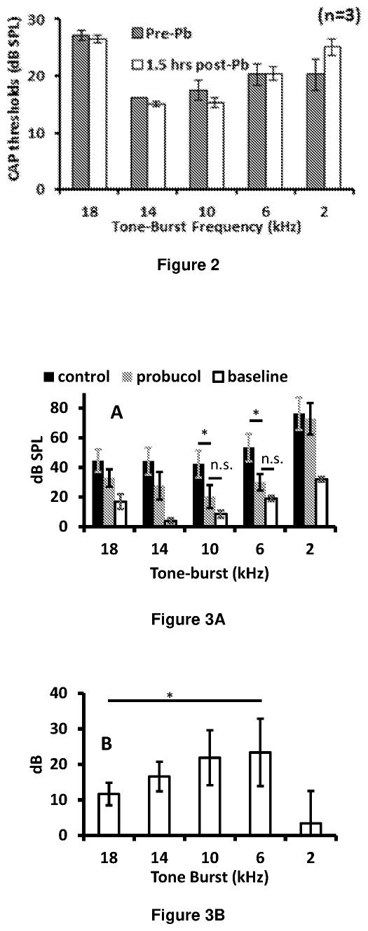

The results are shown in FIG. 2. The shaded columns show the average baseline (pre-probucol) CAP thresholds, while the unshaded column show the average CAP thresholds 1 to 1.5 hours after the application of probucol with Formulation Example 2. The CAP thresholds are given relative to the sound system levels, with an increase in Y axis values representing an improvement in cochlear sensitivity to sound. The experiments were repeated and showed consistent results. There was a non-significant yet visible improvement of CAP thresholds (unshaded column). The formulation did not damage or compromise hearing, thus, the formulation is not acutely ototoxic when injected directly into the round window of anaesthetised healthy rats.

Any suitable materials and/or methods known to those of skill can be utilized in carrying out the present invention. However, preferred materials and methods have been described. Materials, reagents and the like to which reference are made herein in the description and examples are obtainable from commercial sources, unless otherwise noted.

### Experimental Example 3: Protective Effects of Formulation Example 1 on Hearing

Experimental Example 3 demonstrates the significant protective effects of Formulation Example 1 on hearing.

Experiments were performed on nine adult guinea pigs (Cavia porcellus) of either sex with body weights between 250 g to 350 g. All experimental procedures were approved by Curtin University's Curtin Animal Ethics Committee (AEC #2020-15). For all experiments each animal first underwent a recovery surgery at day ‘zero’ for the purposes of: (1) obtaining baseline Compound Action Potential (CAP) thresholds, via a silver chloride (AgCl) ball electrode placed on the round window of the cochlea (with the reference electrode in the neck musculature), in response to 2, 6, 10, 14 and 18 kHz tone bursts of 5 ms duration; (2) surgically exposing the middle ear to apply the oto-protective drug to the round window membrane, after baseline CAP thresholds had been obtained; and (3) inducing ototoxicity via a single dose of both kanamycin (400 mg/kg, s.c., GoldBio©, St. Louis USA; injected immediately after CAP threshold measurements) and furosemide (100 mg/kg, i.v., Centrafarm Pharmaceuticals, the Netherlands; injected via the external jugular vein 20-30 minutes after kanamycin injection). Prior to anaesthesia, each animal was given a 0.05 mg/kg s.c. injection of Temgesic (Buprenorphrine; Reckitt Benckiser, Auckland NZ) and 0.1 ml s.c. injection of Atrosine (0.6 mg/ml atropine sulphate; Apex Laboratories, NSW, Australia) as a pre-med, before being anesthetized using isoflurane (2-5%). The temperature was maintained using an electric heating pad and rectal temperature probe, and lignocaine was used as a local anaesthetic.

While using aseptic techniques, animals were mounted on a custom ear-bar setup, and the right bulla was surgically opened via a dorsal approach providing a view of the right round window, using aseptic techniques. Once the round window was exposed, electrodes were placed for CAP recordings, and then subsequently removed, for the placement of the probucol-gel. Approximately 100 μL of the gel was placed over the entire round window membrane, and whilst attempts were made to prevent movement of the gel to the oval window, it was acknowledged that this may have occurred in some instances due to the small middle ear space. The bulla opening was partially resealed using bone-fragments, before the overlying skin was sutured. The animal was then removed from isoflurane, and allowed to recover slowly under close observation. During recovery, the animal was given an s.c. injection of meloxicam (5 mg/kg s.c.; Troy Laboratories New Zealand) and, on occasions, some animals were given a subsequent 0.05 mg/kg s.c. dose of Temgesic. Animals were then allowed to recover for 18 days (±4 days). Following the recovery period, animals were re-anaesthetized and mounted on the ear-bars, the bulla was re-opened, and a repeated measure of CAP thresholds was performed from both cochleae. Animals were then deeply anaesthetized, and euthanized via cardiac fixation for temporal bones harvesting.

The primary functional measure of cochlear sensitivity was the lowest sound level (in dB SPL) at which a CAP response could be evoked by the five different frequencies. This was determined by presenting each tone burst at a range of sound levels, and plotting a linear trendline to the CAP growth response, and determining the sound level at which a CAP of zero amplitude would have been evoked.

Secondary measures included a histological examination of the cochlea post-mortem, which were imaged using a custom-designed light sheet fluorescence microscope. Here, spiral ganglion cell counts were made in the apical, middle, and basal turns of the cochlea.

### Results:

Using the non-probucol treated cochlea as a control ear, and comparing the CAP thresholds to the baseline measures, the kanamycin and furosemide treatment resulted in a significant (p<0.05; one-tailed t-test assuming equal variances) 24-45 dB hearing loss across all five test frequencies (FIG. 3A). The probucol-treated cochleae demonstrated an 11-40 dB hearing loss compared to the baseline measures, which was significant at all frequencies except for 6 and 10 kHz (FIG. 3A). Compared to the non-probucol treated cochlea, the average probucol-treated cochlear CAP threshold was significantly lower (i.e. better) at 6 kHz and 10 kHz (FIG. 3A). These results are based on the average CAP thresholds for each group (baseline, control, or probucol-treated), but given that each animal acted effectively as its own control, it was possible to examine the mean difference between left and right CAP thresholds, for each animal, at each frequency (FIG. 38). Here, there was a significant (p<0.05) 11 to 23 dB difference for 6, 10, 14 and 18 kHz evoked CAP thresholds, with the probucol-treated cochlea exhibiting a lower CAP threshold, but an insignificant 3 dB difference at 2 kHz.

### Experimental Example 4: Otoprotective Effect of Probucol (PB) for Ototoxicity Induced Hearing Loss in Guinea Pig

Experimental Example 4 describes a nanoparticulate system using probucol. The nanoparticulate system is based on a nanocapsule of hydrogel with probucol (PB) with 2-hydroxypropyl-β-cyclodextrin (CDX), and deoxycholic acid (DCA).

Hydrogels with PB were prepared using deoxycholic acid and 2-hydroxypropyl-β-cyclodextrin to further enhance biocompatibility, penetration rates of RWM (round window membrane). The gel including PB is a selective drug that prevents hearing loss from occurring prior to the onset of hearing loss due to ototoxicity. Therefore, the experiment shows that the gel loaded with probucol injected on the RWM is useful as a tool for enhancing the therapeutic effect of ototoxic hearing loss diseases of the inner ear.

### Materials and Methods

### Reagents and Excipients:

Probucol (PB), 2-hydroxypropyl-β-cyclodextrin (CDX), deoxycholic acid (DCA), polysorbate 80 (Tween 80), propylene glycol (PG) and low viscosity sodium alginate (LVSA) where all purchased from Sigma-Aldrich Australia (Castle Hill, NSW, Australia). Metron® Water soluble gel (WSG) was purchased from Australian Medical Association (Perth, WA, Australia). MilliQ water was acquired via automated Merck Millipore Filtration system.

### Nanocapsule Formation and Hydrogel Delivery System:

Nanocapsules loaded with three different concentrations of PB were made to determine the pharmacodynamic and toxicology profile of the formulation system. The final concentration of PB in the nanocapsule-hydrogel formulation system were 25, 50 and 75% w/w.

### (1) Stock Solutions

Hydrogel matrix was made by dissolving WSG in MilliQ water at 100 mg/mL and heating at 75° C. (stirred at 2500 RPM) for 25 minutes. LVSA stock solution was made by dissolving the powder in MilliQ water at 1.5% w/v according to established protocols (55° C. at 2000 RPM). A co-solvent polymer-hydrogel matrix system was made by adding the WSG mixture to the LVSA mixture at a ratio of 3:1, heating at 58° C. for 25 minutes and stirring at 3000 RPM.

### (2) Nanocapsule Formation and Hydrogel Delivery Matrix

PB-DCA nanocapsules were made via in situ capsular complexation chemistry. This method deploys the inherent capacity of CDX to complex, engulf and effectively encapsulate non-polar, highly lipophilic compounds within their core while being easily dispersed in a co-solvent hydrogel-polymer matrix due to their hydrophilic outer shell. To achieve encapsulation, three identical mixtures CDX in hydrogel-polymer matrix system were made. Firstly, CDX was uniformly dispersed 2.5 g in 100 mL aliquot of the previously made hydrogel-polymer co-solvent formulation delivery matrix at 80° C. for 35 minutes (3000 RPM). PB was added to each of the three mixtures at three different concentrations of 25% w/w (nanocapsules A), 50% w/w (nanocapsules B), at 75% w/w (nanocapsules C). The mixtures were allowed to stir at 80° C. for 3 hours at 2000 RPM. This allowed sufficient time for uniform incorporation of PB within the CDX core, creating nanocapsular PB-loaded drug delivery systems within the hydrogel-polymer matrix. DCA was added at 2% w/w to each formulation (A, B and C) and stirred at 2000 RPM for 35 minutes at 80° C. To each formulation, 1.5 mL of Tween 80 and 1 mL of propylene glycol was added, mixed for 10 minutes at 2000 RPM at 80° C. The formulations were allowed to cool to room temperature prior to administration for the purpose of animal studies.

### Ototoxicity in the HEI-OC1 Cell

House Ear Institute-Organ of Corti 1 (HEI-OC1) cells provided by Y J. Seo (Yonsei University, wonju, Korea) and F. Kalinec, (House Ear Institute, (Los Angeles, Calif., USA) were cultured in high-glucose Dulbecco's modified Eagle's medium (DMEM; Lonza, USA) supplemented with 10% Fetal bovine serum (FBS; Gibco BRL, USA) and 50 U/ml gamma-interferon (PEPRO TECH, INC. USA) without any antibiotics in a permissive condition (33° C., 10% CO₂). For the non-permissive condition, cultured cells were cultured in the permissive condition for a certain period (Kalinec G, Thein P, Park C, Kalinec F. HEI-OC1 cells as a model for investigating drug cytotoxicity. Hearing Res. 2016; 335:105-117. doi:10.1016/j.heares.2016.02.019). Growth curves were performed to identify the growth patterns of cells in each condition. Cells cultured at 33° C., in a 10% CO₂ atmosphere condition were recorded on day 0 after cell counting before seeding. Cells were recovered stripped daily from the cell dish and enumerated. This was done then, cell counted, confirmed for up to 14 days. Following the shift, cells were enumerated daily for 14 days. As explained above, HEI-OC1 cells were cultured in permissive conditions (33° C., 10% CO₂) until day 5 and then transferred to the non-permissive condition (39° C., 5% CO₂).

Cisplatin was from Sigma Chemical Co. (St. Louis, Mo., USA). Probucol-gel drug treatment was used for 24 hours in cisplatin induced ototoxicity: untreated (control) and treated with cisplatin soluble in DMSO (both from Sigma). In preliminary experiments, the CCK assay was performed as described next to confirm if the treatment concentration affected viability. Almost no cell death was observed with 1 mM cisplatin. Subsequent experiments used 20 μM cisplatin to ensure IC 50 on cell viability. High-glucose DMEM supplemented with 0.5% FBS was used.

Ototoxicity Animal Model with Kanamycin and Furosemide

Experiments were performed on 9 adult guinea pigs (Cavia porcellus) of either sex with body weights between 250 and 350 g. All experimental procedures were approved by The University of Curtin Animal Ethics Committee.

For all experiments, each animal first underwent a recovery surgery at day ‘zero’ to inject.

For the recovery surgery, each animal was given a 0.05 ml subcutaneous injection of Temgesic (Buprenorpherine; Reckitt Benckiser, Auckland NZ) and 0.1 ml of Atrosine (0.6 mg/ml atropine sulphate; Apex Laboratories, NSW, Australia) before being anesthetized using isoflurane (2-5%). The temperature was maintained using an electric heating pad, and lignocaine was used as a local anesthetic. While using aseptic techniques, animals were mounted on a custom ear-bar setup, and the left bulla surgically opened via a dorsal approach providing a view of the left round window, using aseptic techniques. Before the drug administrations, the AgCl ball-electrode (reference in the neck) was placed on the round window for recording compound action potential (CAP) responses (Brown D J, Chihara Y, Curthoys I S, Wang Y, Bos M. Changes in cochlear function during acute endolymphatic hydrops development in guinea pigs. Hearing Res. 2013; 296 (J. Neurophysiol. 101 2009):96-106. doi:10.1016/j.heares.2012.12.004; Brown D J, Pastras C J, Curthoys I S, Southwell C S, Roon L V. Endolymph movement visualized with light sheet fluorescence microscopy in an acute hydrops model. Hearing Res. 2016; 339 (PloS one 7 2012):112-124. doi:10.1016/j.heares.2016.06.007; and Brown D J, Sokolic L, Fung A, Pastras C J. Response of the Inner Ear to Lipopolysaccharide Introduced Directly into Scala Media. Hearing Res. 2018; 370 (Otol Neurotol 32 2011):105-112. doi:10.1016/j.heares.2018.10.007).

The ototoxic treatment protocols were performed with kanamycin and furosemide respect to vestibular and cochlear function. All of them received a single dose of both kanamycin (400 mg/kg, s.c.) and furosemide (100 mg/kg, i.v.) on the recovery surgery day. The vena jugularis externa was exposed and cannulated and a single dose of furosemide (Centrafarm Pharmaceuticals, Etten-Leur, The Netherlands) was slowly infused at a dose of 50 mg/kg body weight. The Probucol-gel was administered on the round window though intra-bulla at an amount that covered the round window enough. The bulla opening was resealed with dental cement before the overlying skin was sutured. The animal was then removed from isoflurane, and allowed to recover slowly under close observation. During recovery, the animal was given a subsequent 0.05 ml i.p. dose of Temgesic, along with an oral delivery of 1 ml Ibuprofen (40 mg/ml; Reckitt Benckiser Pty Ltd, Australia). Animals were then allowed to recover for 14 days (±3) days. For following CAP threshold measurements after 14 days, the bulla was re-opened with same procedures. Thereafter, animals were re-anaesthetized and mounted on the ear-bars, for a repeated measure of CAP thresholds described previously. Animals were then deeply anaesthetized, and euthanized via cardiac fixation for temporal bones harvesting.

### Clearing of Cochlea and Light Sheet Fluorescent Microscopy (LSFM) and Quantitative Analyses

Harvested temporal bones were immersed in 4% para-formaldehyde overnight at 4° C., before being decalcified for 14-18 days in 10% EDTA. They were then rinsed with Sorenson's buffer, dehydrated in a series of 50, 75, 90 and 100% ethanol (8-12 h each step), and immersed in Spalteholz solution for 24 h, to render the sample optically transparent. Samples were then imaged using a custom light-sheet fluorescence microscope (LSFM), based on the setup described by Santi et al. (2009) (Santi P A. Light Sheet Fluorescence Microscopy. J Histochem Cytochem. 2010; 59(2):129-138. doi:10.1369/0022155410394857), where ‘virtual slices’ of the inner ear were imaged at 5 mm steps in the axial plane, detailed in Brown et al. (2016) (Brown D J, Pastras C J, Curthoys I S, Southwell C S, Roon L V. Endolymph movement visualized with light sheet fluorescence microscopy in an acute hydrops model. Hearing Res. 2016; 339 (PloS one 7 2012):112-124. doi:10.1016/j.heares.2016.06.007), providing a ‘z-stack’ of between 300 and 1000 virtual slice images. LSFM images were analyzed by a blinded investigator, for later comparison to the functional data and treatment. LSFM z-stacks were analyzed using ImageJ software, with 3D volumes segmented for volume analysis using the Segmentation3D plugin. TIFF files collected from the light sheet microscope were reconstructed into representative 3D images. Using the Surfaces tool of the Arivis vision4D software, the cochlea and SGNs volumes were determined separately, based on thresholds corresponding to their respective autofluorescence intensities (Brown D J, Pastras C J, Curthoys I S, Southwell C S, Roon L V. Endolymph movement visualized with light sheet fluorescence microscopy in an acute hydrops model. Hearing Res. 2016; 339 (PloS one 7 2012):112-124. doi:10.1016/j.heares.2016.06.007).

### Immunofluorescence

Immunofluorescence was determined proceeded both in vitro. In vitro, cells were cultured at 33° C. in 4-well dishes. After culturing in a 4-well dish for 24 hours, each sample was washed with 1 ml of PBS and, add 500 μl of 4% paraformaldehyde was added to each well and was fixation proceeded for 1 hour. The paraformaldehyde was removed, and each sample was washed three Remove all 4% PFA, wash 3 times for 5 minutes each in PBS. After the final wash, add 500 μl of 0.1% Triton X-100 was added and incubated for 15 minutes at room temperature (RT). Triton X-100 was removed and, samples were washed three times for 5 minutes each in PBS. The primary antibody was diluted 1:100 in 5% Normal Goat Serum (NGS) GS was added and the cells were incubated for incubate 1 hour at RT. Mysoin and nestin were used as the primary antibody. After 1 hour, PBS (500 μl) was then added to each well and washed three times (5 minutes per wash). (500 μl) and washed 3 times for 5 minutes. Secondary antibody was used Alexa488-Rabbit (ab150077, Abcam, UK). After 1 hour, each sample was mounted using 4′,6-diamidino-2-phenylindole (DAPI) and was covered with a cover glass. The solution was dried for 24 hours in a dark light-blocked room and examined using confirmed by confocal microscopy.

### Statistical Analysis

SPSS statistics (IBM SPSS Statistics for Windows, V.24, Armonk, NY) software was used for all statistical analysis. Descriptive results of continuous variables in cell study are expressed as the mean±standard deviation (SD) for normally distributed variables. Means were compared by 2-way analysis of variance. The level of statistical significance was set to 0.05. Changes in CAP thresholds at day 1, 7 and 42 (relative to day zero), were compared across treatment groups for all tone-burst frequencies using a one-way MANOVA, with Tukey's post-hoc analysis.

### Results

### Probucol Effect In Vitro

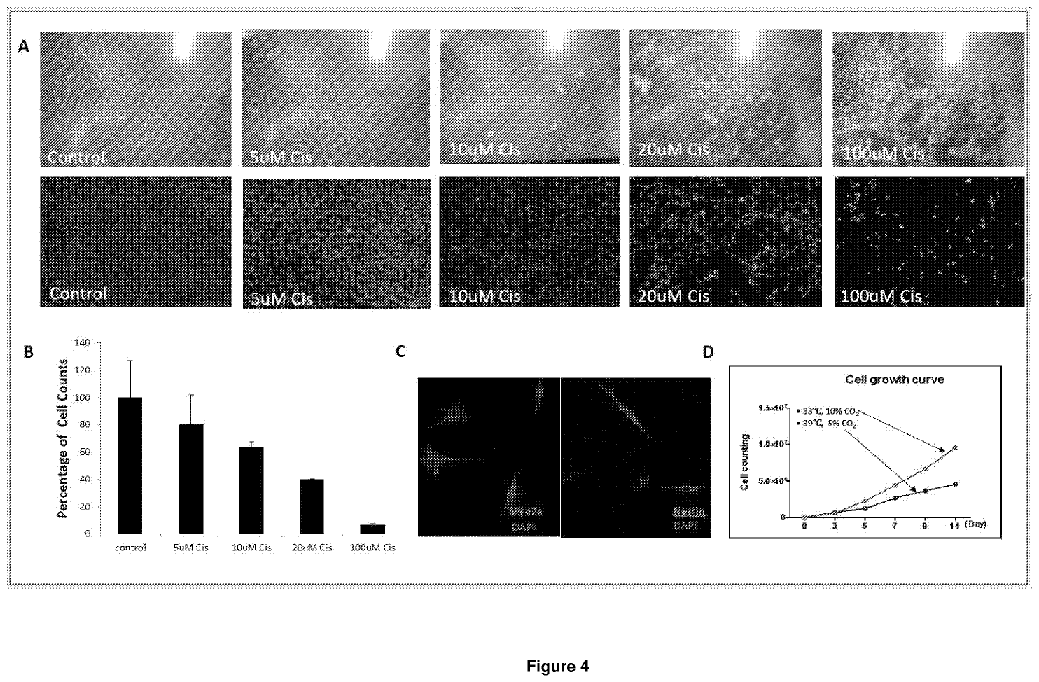

House Ear Institute-Organ of Corti 1 (HEI-OC1) cells are derived from the auditory organ of a transgenic mouse. Because it is difficult to keep it cleaned, it was necessary to confirm whether the HEI-OC1 cells in the expressions of Myosin7A and Nestin well as hair cell markers and the normal growth pattern (FIGS. 4C and 4D) before the cytotoxicity.

To induce the ototoxicity in the inner ear hair cells (HEI-OC1), cells were seeded in culture plates in equal numbers and left to grow to 80% confluence in a medium containing 10% FBS. Cells were washed twice with PBS buffer and maintained in medium containing cisplatin at the concentrations of 5-100 μM for 24 h. As shown in FIG. 4, raising the ambient cisplatin concentration (5, 10, 20, 100 μM) causes a dose-dependent decrease in cell viability (up to 8% at 100 uM cisplatin when compared with control).

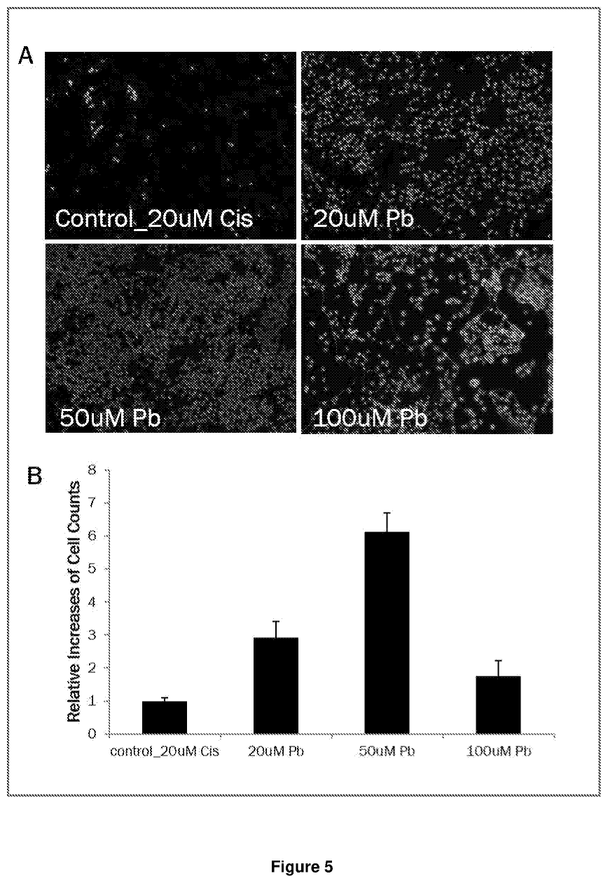

HEI-OC1 cells were cultured in medium containing cisplatin and probucol for 24 h to determine whether probucol modulated oto-protection. Then the cells treated simultaneously with probucol or probucol's vehicle (0.05% DMSO) for 24 h showed the protection (FIG. 5). It was found that probucol (at 20 μM and 50 μM) promoted the cell survival when compared with control. At the 50 μM of probucol, it showed the protective effect on ototoxicity induced inner ear cells more than 6 times compared to the control. But the high concentration (100 μM) of probucol caused a significant decrease in cellular mitogenesis when compared with control, because it's vehicle, DMSO, also had a high concentration and it affected the cytotoxicity.

### Probucol Effect In Vivo in Hearing

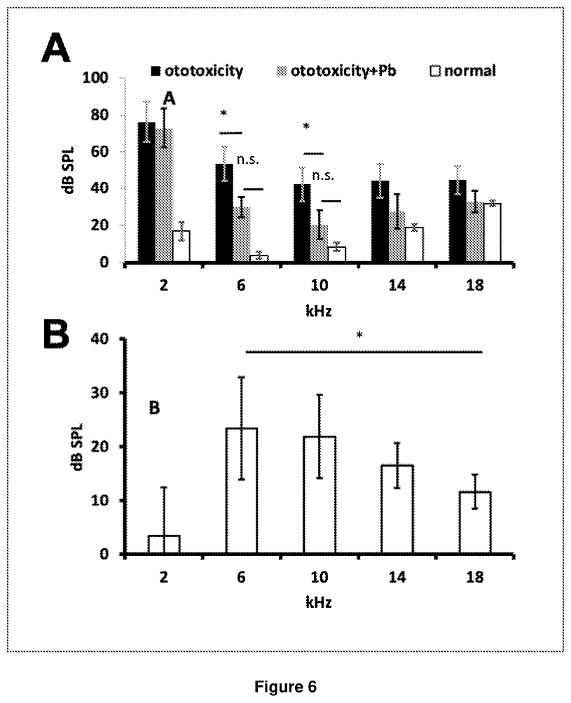

Average change in CAP thresholds (with standard deviation) to tone bursts of 2, 6, 10, 14 and 18 kHz, measured 0 and 14 days after animals had 0.4 ml of probucol injected into the bulla around the RWM (FIG. 5). The standard deviation of CAP thresholds was measured at day 0. CAP thresholds on the side without probucol injection were elevated by about 40 dB across all frequencies after the ototoxicity with kanamycin and furosemide (FIG. 6). But on day 14 post-treatment, the side with probucol injection into RWM showed not as severe of a shift as that induced by kanamycin and furosemide injections (the differences of CAP between the ototoxicity (left side) and the ototoxicity with probucol (right side) were 23.4±9.49 dB at 6 kHz, 21.89+7.72 at 10 kHz, and 16.56±4.15 at 14 kHz). But the CAP showed that the side treated with probucol presented better thresholds of hearing than those of the control side, especially at high frequencies significantly (only 6 and 10 kHz).

### Probucol Effect in Light Sheet Microscopy (LSM) Images

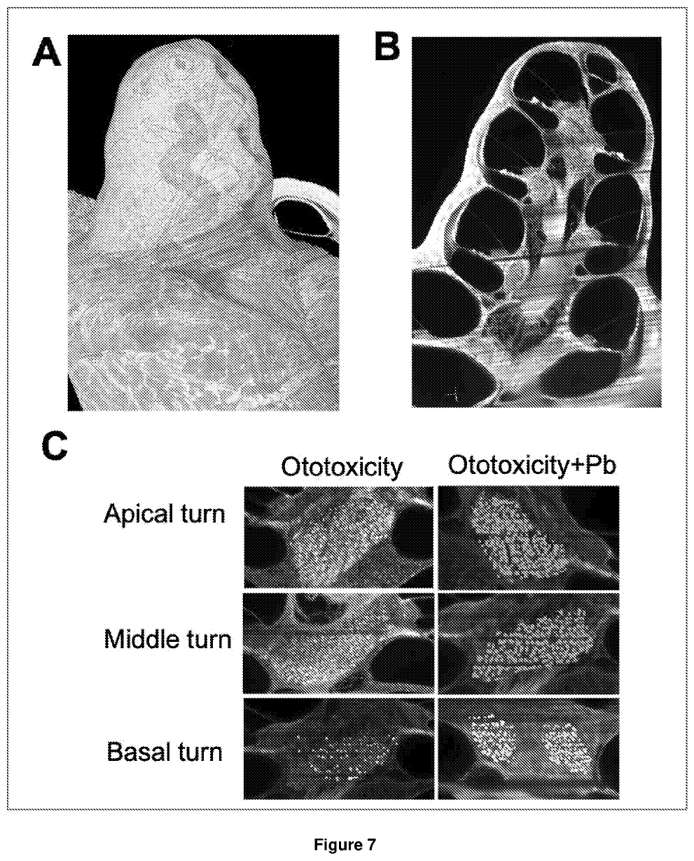

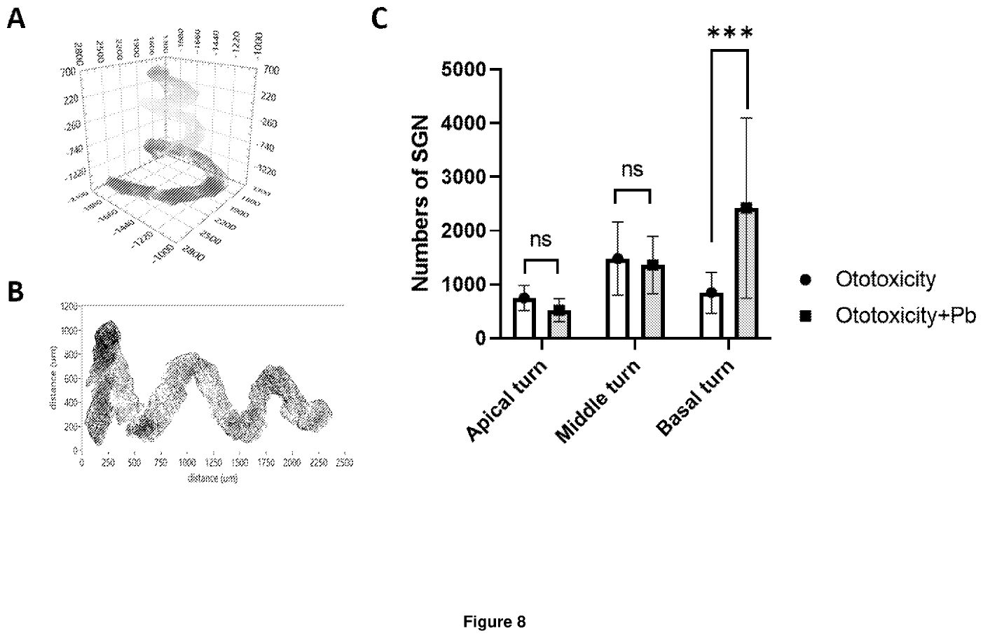

The data acquired by LSM can allow extraction and quantification of several organoid features, including cell number at the micro- and mesoscale via our validated nuclei segmentation. The inventors intended to generate a complete three-dimensional representation of the spiral ganglion neuron (SGN) as it ascends from base to apex in the intact cochlea. The 2D sectional images might give limited information of SGNs in the whole cochlea. The inventors next quantified the density of SGNs within LSM-imaged cochlea using Arivis Vision4D software's ‘blob analysis’ tool by rendering the volume surfaces of the entire SGNs (FIG. 7). In the FIG. 7C, the blobs included every SGNs on 2D images, because the yellow blobs skipped the overlapped SNGs around close section slides. As expected, there was less density of SGNs on the basal turn with ototoxicity than the other two turns (middle and apical). Next, the inventors analyzed the density of SGNs in ototoxicity and probucol group by LSM to identify cellular density (FIG. 8). The density of SGNs on each turn in a cochlea was calculated as the average of the cell populations of the 3 segments (100 μm in a segments) in each turn analyzed according to z-axis. It showed that the cell density is less in the basal turns than those in apical and middle turns of ototoxicity group. But the cell density on the basal turns were kept not to be injured in the probucol treatment group significantly, compared to the control group (ototoxicity). Probucol had a protective effect on hair cell damages by ototoxicity drugs.

### Discussion

In this study, probucol clearly showed a hearing protection effect in the ototoxicity guinea pig model. Thus, the present inventors have shown that probucol may be used for an effective therapeutic drug in hearing loss.

### SPIM Images

LSM uses a thin plane of light to optically section transparent tissues or whole organisms that have been labeled with a fluorophore. Three-dimensional structures in biological systems are routinely evaluated using large image stacks acquired from fluorescence microscopy. LSM is a nondestructive method that produces well-registered optical sections that are suitable for three-dimensional reconstruction and can be processed without any histological sectioning. It is necessary to visualize and assess all array of the hair cells that travel around a central core from the base to the apex of the cochlea because frequencies of sound are mapped from high to low from the cochlear base to the apex. An imaging method in the whole cleared cochlea was introduced by making the bone transparent after fixation and decalcification using a clearing solution (Santi P A. Light Sheet Fluorescence Microscopy. J Histochem Cytochem. 2010; 59(2):129-138. doi:10.1369/0022155410394857). The inventors chose the CUBIC protocol which Susaki et al. developed for its simplicity, low cost, low toxicity, potential compatibility with immunostaining, and good preservation of fluorescent proteins (Susaki E A, Tainaka K, Perrin D, Yukinaga H, Kuno A, Ueda H R. Advanced CUBIC protocols for whole-brain and whole-body clearing and imaging. Nat Protoc. 2015; 10(11):1709-1727. doi:10.1038/nprot.2015.085). CUBIC tissue clearing enabled the inventors to use light sheet imaging to visualize axonal tracts in the intact HVC-RA projection (Rocha M D, Düring D N, Bethge P, et al. Tissue Clearing and Light Sheet Microscopy: Imaging the Unsectioned Adult Zebra Finch Brain at Cellular Resolution. Front Neuroanat. 2019; 13:13. doi:10.3389/fnana.2019.00013). The imaging of autofluorescent structures allowed visualization of cochlea microstructures throughout the intact bone in CUBIC-cleared tissue. Segmentation on 2D images is usually a time-consuming, manual procedure, but this remains a challenging problem with being developed in combination with specific labeling to automate the segmentation process. Current efforts are underway to include the use of automated segmentation and machine learning processes to facilitate automated interpretation and improve quality control measures (Eskandari M, Kramer C M, Hecht H S, Jaber W A, Marwick T H. Evidence Base for Quality Control Activities in Cardiovascular Imaging. Jacc Cardiovasc Imaging. 2016; 9(3):294-305. doi:10.1016/j.jcmg.2015.11.012). In the present study, three-dimensional rendered structures of SGNs autocalculated by blob analysis of Arivis Vision4D was used to visualize their three-dimensional anatomy but also to estimate their morphometric parameters like cell density and volume.

Although the application has been disclosed in the context of certain embodiments and examples, it will be understood by persons skilled in the art that the embodiments of the application extend beyond the specifically disclosed embodiments to other alternative embodiments and/or uses and modifications and equivalents thereof. That is, variations on those embodiments will become apparent to persons skilled in the art upon reading the foregoing description. It is contemplated that the skilled person can employ such variations as appropriate, and the application may be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

Embodiments of the invention are described below in the following items 1 to 33:

1. A method for preventing, reducing or treating the incidence and/or severity of a disorder caused by stress-induced cellular damage in the middle or inner ear of a subject, the method comprising administering to the subject a therapeutically effective amount of probucol.
2. A method for preventing, reducing or treating the incidence and/or severity of a vestibular disorder or hearing impairment in a subject, the method comprising administering to the subject a therapeutically effective amount of probucol.
3. A method for preventing, reducing or inhibiting hair cell degeneration or hair cell death in a subject, the method comprising administering to the subject a therapeutically effective amount of probucol.
4. A method according to any one of items 1 to 3, wherein the probucol is administered by the transtympanic route.
5. A method according to any one of items 1 to 4, wherein the probucol is administered simultaneously, separately or sequentially in combination with a further active ingredient.
6. A method according to item 5, wherein the further active ingredient is selected from a steroid or an ototoxic therapeutic drug.
7. A method according to item 6, wherein the further active ingredient is selected from dexamethasone, methylprednisolone, prednisolone, and gentamicin.
8. A method according to any one of items 1 to 7, wherein the probucol is administered daily.
9. A method according to any one of items 1 to 8, wherein the probucol is administered one or more times a day.
10. A method according to any one of items 1 to 9, wherein the probucol is administered in a formulation comprising:
(i) probucol;
(ii) an agent that enhances the bioavailability of the active ingredient, wherein the agent comprises an amphiphilic compound of the formula (I):

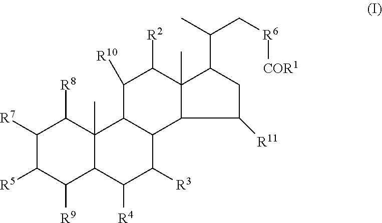

wherein:
   each R¹, R², R³, R⁴, R⁵, R⁷, R⁸, R⁹, R¹⁰ and R¹¹ is independently selected from H, substituted or unsubstituted C₁₋₃₀ alkyl, substituted or unsubstituted C₂₋₃₀ alkenyl, substituted or unsubstituted C₂₋₃₀ alkynyl, substituted or unsubstituted C₃₋₃₀ cycloalkyl, substituted or unsubstituted aryl, substituted or unsubstituted arylalkyl, substituted or unsubstituted heteroaryl, or GL; wherein when R¹, R², R³, R⁴, R⁵, R⁷, R⁸, R⁹, R¹⁰ or R¹¹ is substituted, the substituent is independently selected from F, OH, SH, ═O, ═S, Cl, Br, SC₁₋₆ alkyl, C₁₋₆ alkyl or C₁₋₆ alkoxy;
   G is absent or is selected from O, S, PL, CL₂, or NL;
   each L is independently selected from H, a metallic ion, substituted or unsubstituted C₁₋₃₀ alkyl, substituted or unsubstituted C₂₋₃₀ alkenyl, substituted or unsubstituted C₂₋₃₀ alkynyl, substituted or unsubstituted C₃₋₃₀ cycloalkyl, a substituted or unsubstituted benzyl radical, —CH₂CO₂H, or —(CH₂)₂SO₃H; wherein when L is substituted, the substituent is independently selected from F, OH, SH, ═O, ═S, Cl, Br, SC₁₋₆ alkyl, C₁₋₆ alkyl or C₁₋₆ alkoxy; or when L is bonded to R¹, L may be an amino acid; and
   R⁸ is —(CH₂)ₙ— wherein n is 0 to 12;
   or a stereoisomer thereof, or a pharmaceutically acceptable salt thereof; and
(iii) optionally a pharmaceutically acceptable carrier.
   11. Use of probucol in the manufacture of a medicament for preventing, reducing or treating the incidence and/or severity of a disorder caused by stress-induced cellular damage in the middle or inner ear of a subject.
   12. Use of probucol in the manufacture of a medicament for preventing, reducing or treating the incidence and/or severity of a vestibular disorder or hearing impairment in a subject.
   13. Use of probucol in the manufacture of a medicament for preventing, reducing or inhibiting hair cell degeneration or hair cell death in a subject.
   14. Use according to any one of items 11 to 13, wherein medicament is formulated for administration by the transtympanic route.
   15. Probucol for use in preventing, reducing or treating the incidence and/or severity of a disorder caused by stress-induced cellular damage in the middle or inner ear of a subject.
   16. Probucol for use in preventing, reducing or treating the incidence and/or severity of a vestibular disorder or hearing impairment in a subject.
   17. Probucol for use in preventing, reducing or inhibiting hair cell degeneration or hair cell death in a subject.
   18. A pharmaceutical formulation comprising:
(i) an active ingredient;
(ii) an agent that enhances the bioavailability of the active ingredient, wherein the agent comprises an amphiphilic compound of the formula (I):

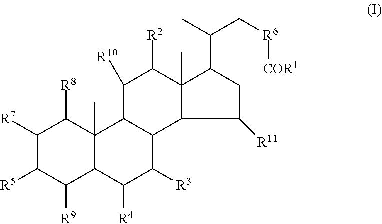

wherein:
   each R¹, R², R³, R⁴, R⁵, R⁷, R⁸, R⁹, R¹⁰ and R¹¹ is independently selected from H, substituted or unsubstituted C₁₋₃₀ alkyl, substituted or unsubstituted C₂₋₃₀ alkenyl, substituted or unsubstituted C₂₋₃₀ alkynyl, substituted or unsubstituted C₃₋₃₀ cycloalkyl, substituted or unsubstituted aryl, substituted or unsubstituted arylalkyl, substituted or unsubstituted heteroaryl, or GL; wherein when R¹, R², R³, R⁴, R⁵, R⁷, R⁸, R⁹, R¹⁰ or R¹¹ is substituted, the substituent is independently selected from F, OH, SH, ═O, ═S, Cl, Br, SC₁₋₆ alkyl, C₁₋₆ alkyl or C₁₋₆ alkoxy;
   G is absent or is selected from O, S, PL, CL₂, or NL;
   each L is independently selected from H, a metallic ion, substituted or unsubstituted C₁₋₃₀ alkyl, substituted or unsubstituted C₂₋₃₀ alkenyl, substituted or unsubstituted C₂₋₃₀ alkynyl, substituted or unsubstituted C₃₋₃₀ cycloalkyl, a substituted or unsubstituted benzyl radical, —CH₂CO₂H, or —(CH₂)₂SO₃H; wherein when L is substituted, the substituent is independently selected from OH, F, Cl, SH, ═O, ═S, Br, SC₁₋₆ alkyl, C₁₋₆ alkyl or C₁₋₆ alkoxy; or when L is bonded to R¹, L may be an amino acid; and
   R⁸ is —(CH₂)ₙ— wherein n is 0 to 12;
   or a stereoisomer thereof, or a pharmaceutically acceptable salt thereof; and
(iii) optionally a pharmaceutically acceptable carrier.
   19. A formulation according to item 18, wherein the amphiphilic compound of the formula (I) is selected from deoxycholic acid, cholic acid, taurocholic acid, glycocholic acid, glycodeoxycholic acid, taurodeoxycholic acid, ursodeoxycholic acid, taurochenodeoxycholic acid, lithocholic acid, glycolithocholic acid, chenodeoxycholic acid, taurolithocholic acid, tauroursodeoxycholic acid, obeticholic acid, α-muricholic acid, β-muricholic acid, γ-muricholic acid, ω-muricholic acid, glycomuricholic acid, tauromuricholic acid, and glycochenodeoxycholic acid, or a salt, derivative or metabolite thereof.
   20. A formulation according to item 18 or 19, wherein the pharmaceutically acceptable carrier is selected from glycerol, propylene glycol, polysorbate 80 (Tween 80), gels, hyaluronic acid, polyvinyl alcohol (PVA), poly-lactic-co-glycolic acid (PLGA), and PEG400, or a combination thereof.
   21. A formulation according to any one of items 18 to 20, wherein the active ingredient is probucol.
   22. A formulation according to according to any one of items 18 to 21, wherein the formulation is in the form of a solution, a suspension, microparticles, nanoparticles, or a gel.
   23. A formulation according to any one of items 18 to 22, wherein the formulation comprises 0.1 to 50% w/w of probucol.
   24. A method for preventing, reducing or treating the incidence and/or severity of a disorder caused by stress-induced cellular damage in the middle or inner ear of a subject, the method comprising administering to the subject an effective amount of the formulation according to any one of items 21 to 23.
   25. A method for preventing, reducing or treating the incidence and/or severity of a vestibular disorder or hearing impairment in a subject, the method comprising administering to the subject an effective amount of the formulation according to any one of items 21 to 23.
   26. A method for preventing, reducing or inhibiting hair cell degeneration or hair cell death in a subject, the method comprising administering to the subject an effective amount of the formulation according to any one of items 21 to 23.
   27. Use of probucol in the preparation of a formulation according to any one of items 21 to 23 for preventing, reducing or treating the incidence and/or severity of a disorder caused by stress-induced cellular damage in the middle or inner ear of a subject.
   28. Use of probucol in the preparation of a formulation according to any one of items 21 to 23 for preventing, reducing or treating the incidence and/or severity of a vestibular disorder or hearing impairment in a subject.
   29. Use of probucol in the preparation of a formulation according to any one of items 21 to 23 for preventing, reducing or inhibiting hair cell degeneration or hair cell death in a subject.
   30. A method according to any one of items 24 to 26, or the use according to any one of items 27 to 29, wherein the formulation is administered by the transtympanic route.
   31. A formulation according to any one of items 21 to 23 for use in preventing, reducing or treating the incidence and/or severity of a disorder caused by stress-induced cellular damage in the middle or inner ear of a subject.
   32. A formulation according to any one of items 21 to 23 for use in preventing, reducing or treating the incidence and/or severity of a vestibular disorder or hearing impairment in a subject.
   33. A formulation according to any one of items 21 to 23 for use in preventing, reducing or inhibiting hair cell degeneration or hair cell death in a subject.

1-42. (canceled)

43. A method for preventing, reducing or treating the incidence and/or severity of a disorder caused by stress-induced cellular damage in the middle or inner ear of a subject or the incidence and/or severity of a vestibular disorder or hearing impairment in a subject, or for preventing, reducing or inhibiting hair cell degeneration or hair cell death in a subject, the method comprising administering to the subject a therapeutically effective amount of a compound selected from an antioxidant, an anti-inflammatory agent, and an epithelial cell protective agent.

44. The method according to claim 43, wherein the method is for preventing, reducing or treating the incidence and/or severity of a disorder caused by stress-induced cellular damage in the middle or inner ear of a subject.

45. The method according to claim 43, wherein the method is for preventing, reducing or treating the incidence and/or severity of a vestibular disorder or hearing impairment in a subject.

46. The method according to claim 43, wherein the method is for preventing, reducing or inhibiting hair cell degeneration or hair cell death in a subject.

47. The method according to claim 43, wherein the compound is selected from probucol and succinobucol (4-[2,6-ditert-butyl-4-[2-(3,5-ditert-butyl-4-hydroxyphenyl)sulfanylpropan-2-ylsulfanyl]phenoxy]-4-oxobutanoic acid).

48. The method according to claim 43, wherein the compound is probucol.

49. The method according to claim 43, wherein the compound is administered by the transtympanic route.

50. The method according to claim 43, wherein the compound is administered simultaneously, separately or sequentially in combination with a further active ingredient.

51. The method according to claim 50, wherein the further active ingredient is selected from a steroid or an ototoxic therapeutic drug.

52. The method according to claim 43, wherein the compound is administered in a formulation comprising:
(i) the compound;
(ii) an agent that enhances the bioavailability of the compound, wherein the agent comprises an amphiphilic compound of the formula (I): 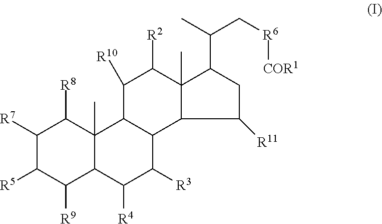
wherein:
each R¹, R², R³, R⁴, R⁵, R⁷, R⁸, R⁹, R¹⁰ and R¹¹ is independently selected from H, substituted or unsubstituted C₁₋₃₀ alkyl, substituted or unsubstituted C₂₋₃₀ alkenyl, substituted or unsubstituted C₂₋₃₀ alkynyl, substituted or unsubstituted C₃₋₃₀ cycloalkyl, substituted or unsubstituted aryl, substituted or unsubstituted arylalkyl, substituted or unsubstituted heteroaryl, or GL; wherein when R¹, R², R³, R⁴, R⁵, R⁷, R⁸, R⁹, R¹⁰ or R¹¹ is substituted, the substituent is independently selected from OH, F, SH, ═O, ═S, Cl, Br, SC₁₋₆ alkyl, C₁₋₆ alkyl or C₁₋₆ alkoxy;
G is absent or is selected from O, S, PL, CL₂, or NL;
each L is independently selected from H, a metallic ion, substituted or unsubstituted C₁₋₃₀ alkyl, substituted or unsubstituted C₂₋₃₀ alkenyl, substituted or unsubstituted C₂₋₃₀ alkynyl, substituted or unsubstituted C₃₋₃₀ cycloalkyl, a substituted or unsubstituted benzyl radical, —CH₂CO₂H, or —(CH₂)₂SO₃H; wherein when L is substituted, the substituent is independently selected from OH, F, SH, ═O, ═S, Cl, Br, SC₁₋₆ alkyl, C₁₋₆ alkyl or C₁₋₆ alkoxy; or when L is bonded to R¹, L may be an amino acid; and
R⁶ is —(CH₂)ₙ— wherein n is 0 to 12;
or a stereoisomer thereof, or a pharmaceutically acceptable salt thereof; and
(iii) optionally a pharmaceutically acceptable carrier.

53. The method according to claim 52, wherein the compound at (i) is selected from probucol and succinobucol (4-[2,6-ditert-butyl-4-[2-(3,5-ditert-butyl-4-hydroxyphenyl)sulfanylpropan-2-ylsulfanyl]phenoxy]-4-oxobutanoic acid).

54. The method according to claim 52, wherein the compound at (i) is probucol.

55. The method according to claim 52, wherein the amphiphilic compound of the formula (I) is selected from deoxycholic acid, cholic acid, taurocholic acid, glycocholic acid, glycodeoxycholic acid, taurodeoxycholic acid, ursodeoxycholic acid, taurochenodeoxycholic acid, lithocholic acid, glycolithocholic acid, chenodeoxycholic acid, taurolithocholic acid, tauroursodeoxycholic acid, obeticholic acid, α-muricholic acid, β-muricholic acid, γ-muricholic acid, ω-muricholic acid, glycomuricholic acid, tauromuricholic acid, or glycochenodeoxycholic acid, or a salt, derivative or metabolite thereof.

56. The method according to claim 52, wherein the pharmaceutically acceptable carrier is selected from glycerol, propylene glycol, polysorbate 80 (Tween 80), gels, hyaluronic acid, polyvinyl alcohol (PVA), poly-lactic-co-glycolic acid (PLGA), and PEG400, or a combination thereof.

57. The method according to claim 52, wherein the formulation is administered by the transtympanic route.

58. The method according to claim 52, wherein the formulation is in the form of a solution, a suspension, microparticles, nanoparticles, or a gel.

59. The method according to claim 52, wherein the formulation comprises 0.1 to 50% w/w of probucol.

60. The method according to claim 52, wherein the formulation is administered simultaneously, separately or sequentially in combination with a further active ingredient.

61. The method according to claim 60, wherein the further active ingredient is selected from a steroid or an ototoxic therapeutic drug.

62. The method according to claim 52, wherein the formulation comprises
(i) probucol or succinobucol;
(ii) an amphiphilic compound selected from deoxycholic acid, cholic acid, taurocholic acid, glycocholic acid, glycodeoxycholic acid, taurodeoxycholic acid, ursodeoxycholic acid, taurochenodeoxycholic acid, lithocholic acid, glycolithocholic acid, chenodeoxycholic acid, taurolithocholic acid, tauroursodeoxycholic acid, obeticholic acid, α-muricholic acid, β-muricholic acid, γ-muricholic acid, ω-muricholic acid, glycomuricholic acid, tauromuricholic acid, or
glycochenodeoxycholic acid, or a salt, derivative or metabolite thereof; and
(iii) optionally a pharmaceutically acceptable carrier.

63. A pharmaceutical formulation comprising:
(i) an active ingredient;
(ii) an agent that enhances the bioavailability of the active ingredient, wherein the agent comprises an amphiphilic compound of the formula (I): 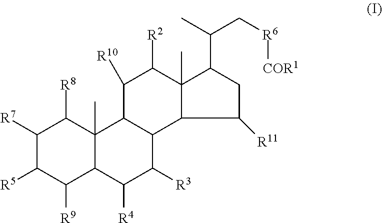
wherein:
each R¹, R², R³, R⁴, R⁵, R⁷, R⁸, R⁹, R¹⁰ and R¹¹ is independently selected from H, substituted or unsubstituted C₁₋₃₀ alkyl, substituted or unsubstituted C₂₋₃₀ alkenyl, substituted or unsubstituted C₂₋₃₀ alkynyl, substituted or unsubstituted C₃₋₃₀ cycloalkyl, substituted or unsubstituted aryl, substituted or unsubstituted arylalkyl, substituted or unsubstituted heteroaryl, or GL; wherein when R¹, R², R³, R⁴, R⁵, R⁷, R⁸, R⁹, R¹⁰ or R¹¹ is substituted, the substituent is independently selected from OH, F, SH, ═O, ═S, Cl, Br, SC₁₋₆ alkyl, C₁₋₆ alkyl or C₁₋₆ alkoxy;
G is absent or is selected from O, S, PL, CL₂, or NL;
each L is independently selected from H, a metallic ion, substituted or unsubstituted C₁₋₃₀ alkyl, substituted or unsubstituted C₂₋₃₀ alkenyl, substituted or unsubstituted C₂₋₃₀ alkynyl, substituted or unsubstituted C₃₋₃₀ cycloalkyl, a substituted or unsubstituted benzyl radical, —CH₂CO₂H, or —(CH₂)₂SO₃H; wherein when L is substituted, the substituent is independently selected from F, SH, OH, ═O, ═S, Cl, Br, SC₁₋₆ alkyl, C₁₋₆ alkyl or C₁₋₆ alkoxy; or when L is bonded to R¹, L may be an amino acid; and
R⁶ is —(CH₂)ₙ— wherein n is 0 to 12;
or a stereoisomer thereof, or a pharmaceutically acceptable salt thereof; and
(iii) optionally a pharmaceutically acceptable carrier;
wherein the formulation is formulated to be administered to the middle or inner ear.

64. The formulation according to claim 63, wherein the formulation is formulated to be administered to the middle or inner ear by the transtympanic route.

65. The formulation according to claim 63, wherein the formulation comprises 0.1 to 50% w/w of probucol.

66. The formulation according to claim 63, wherein the formulation comprises probucol, deoxycholic acid (DCA) and ursodeoxycholic acid (UDCA).

67. The formulation according to claim 66, wherein the formulation is formulated to be administered to the middle or inner ear by the transtympanic route.
